(19) United States
(12) Patent Application Publication
(10) Pub. No.: US 2025/0304622 A1
(43) Pub. Date: Oct. 2, 2025

(54) SHORT APOLIPOPROTEIN E MIMETIC PEPTIDES AND METHODS OF USE

(71) Applicant: The United States of America, as represented by the Secretary, Dept. of Health and Human Services, Bethesda, MD (US)

(72) Inventors: Alan T. Remaley, Bethesda, MD (US); Mart Reimund, Rockville, MD (US); Giorgio T. Graziano, Rockville, MD (US); Denis Sviridov, Rockville, MD (US); Amaury L.P. Dasseux, Bethesda, MD (US)

(73) Assignee: The United States of America, as represented by the Secretary, Dept. of Health and Human Services, Bethesda, MD (US)

(21) Appl. No.: 18/862,905

(22) PCT Filed: May 4, 2023
(86) PCT No.: PCT/US2023/066617

§ 371 Date: Nov. 4, 2024

Related U.S. Application Data
(60) Provisional application No. 63/338,654, filed on May 5, 2022.

(51) Int. Cl. C07K 7/08; A61K 38/00; A61P 3/06

(52) U.S. Cl. CPC C07K 7/08; A61P 3/06; A61K 38/00; C07K 2319/30

(57) ABSTRACT

ApoE mimetic peptides of 8-17 amino acids long including the ApoE receptor binding region or a portion thereof, and one or more covalent linkages joining at least two non-contiguous amino acids of the peptide are provided. Methods of treating dyslipidemic disorders, such as hypertriglyceridemia or hypercholesterolemia, or viral infection using the peptides are also provided.

### CROSS REFERENCE TO RELATED APPLICATIONS

This application claims priority to U.S. Provisional Application No. 63/338,654, filed May 5, 2022, which is incorporated by reference in its entirety.

### FIELD

This disclosure relates to apolipoprotein E (ApoE) mimetic peptides and methods of their use, particularly for treating dyslipidemia or viral infection.

### BACKGROUND

Apolipoprotein E (ApoE) facilitates hepatic clearance of triglyceride-rich lipoprotein remnants by acting as a ligand for the low density lipoprotein receptor (LDLR). In addition, it contains several amphipathic helices and can mediate cholesterol efflux. ApoE includes two structural domains linked by a hinge region. The N-terminal domain includes four alpha-helices. Helix 4 is responsible for binding to receptors and to heparan sulfate proteoglycans. The C-terminal domain includes amphipathic alpha-helices that bind to lipids and also mediate cholesterol efflux by the ABCA1 transporter. Only lipid-bound ApoE binds to LDL-related receptors with high affinity, as the receptor binding regions are shielded in the lipid-free form.

In light of the functions of ApoE, numerous ApoE-mimetic peptides have been designed as potential therapeutics. These have typically included the receptor-binding region and a lipid-binding region (Wolska et al., Cells 10:597, 2021). While some ApoE mimetic peptides are in early clinical trials, there remains a need for ApoE mimetic peptides with improved efficacy, safety, and other properties.

### SUMMARY

Disclosed herein are ApoE mimetic peptides and fusion proteins including the ApoE mimetic peptides. In some examples, the disclosed peptides and fusion proteins have improved properties compared to previous ApoE mimetic peptides.

In some aspects, the ApoE mimetic peptides are 8-17 amino acids in length, include the receptor binding region of ApoE (e.g., SEQ ID NO: 34) or a portion thereof, and include one or more covalent linkages joining at least two non-contiguous amino acids of the peptide. In some examples, the peptide includes an amphipathic helical domain and the covalent linkage joining at least two non-contiguous amino acids is between two amino acids on the hydrophobic side of the amphipathic helical domain. The one or more covalent linkages may be a hydrocarbon staple, a hydrocarbon stitch, a lactam bridge, or a disulfide bond. In some aspects, the covalent linkage is a hydrocarbon staple or hydrocarbon stitch and includes a linkage between one or more of (S)-α-methyl,α-pentenylglycine (S5), (S)-α-methyl,α-octenylglycine (S8), bis-pentenylglycine (B5), (R)-α-methyl,α-pentenylglycine (R5), and (R)-α-methyl,α-octenylglycine (R8), or any combination thereof.

In some aspects, the disclosed ApoE mimetic peptides include one or more additional modifications. In some examples, the modification includes one or more of amino acid substitutions, additions, or deletions; C-terminal amidation; N-terminal acylation; one or more D-isomer amino acids; a modified or non-natural amino acid; an N-terminal fatty acid; a C-terminal fatty acid; or a combination of two or more thereof. In particular examples, the peptide includes an octanoic acid or myristic acid modification at the N- or C-terminus.

In particular examples, the ApoE mimetic peptide includes an amino acid sequence with at least 90% identity to the amino acid sequence of any one of SEQ ID NOs: 2-4, 7-12, 14-21, and 30-33 or includes or consists of the amino acid sequence of any one of SEQ ID NOs: 2-4, 7-12, 14-21, and 30-33.

In some aspects, the ApoE mimetic peptides bind to a lipoprotein (such as one or more of low density lipoprotein (LDL), intermediate-density lipoprotein (IDL), high density lipoprotein (HDL), very low density lipoprotein (VLDL), lipoprotein(a) (Lp(a)), chylomicrons, and chylomicron remnants). In other aspects, the ApoE mimetic peptides bind to heparin or heparan sulfate proteoglycans.

Also provided are fusion proteins that include a disclosed ApoE mimetic peptide, such as an ApoE mimetic peptide that is covalently linked to a second peptide. In some aspects, the fusion protein includes a disclosed ApoE mimetic peptide linked to a human neonatal Fe receptor (FcRn) IgG binding site or FcRn albumin binding site. In some examples, the fusion protein includes an amino acid sequence with at least 90% identity to the amino acid sequence of any one of SEQ ID NOs: 22-25, or the fusion protein includes or consists of the amino acid sequence of any one of SEQ ID NOs: 22-25.

Also provided are pharmaceutical compositions including one or more of the disclosed ApoE mimetic peptides or fusion proteins and a pharmaceutically acceptable carrier. In some aspects, the compositions are formulated for intravenous administration, subcutaneous administration, or oral administration.

Provided herein are methods of treating a subject with a disclosed ApoE mimetic peptide or fusion protein. In some aspects, the methods include treating a subject with dyslipidemia, for example, reducing triglyceride levels in a subject (such as a subject with hypertriglyceridemia) or reducing cholesterol levels in a subject (such as a subject with hypercholesterolemia) by administering an effective amount of a disclosed ApoE mimetic peptide or fusion protein to the subject. In other aspects, the methods include treating a viral infection in a subject (such as a subject with a betacoronavirus infection, for example, SARS-CoV-2 infection) by administering an effective amount of a disclosed ApoE mimetic peptide or fusion protein to the subject. In particular examples, the ApoE mimetic peptide or fusion protein is administered to the subject intravenously, subcutaneously, or orally.

Methods of making the disclosed ApoE mimetic peptides or fusion proteins are also provided. In some aspects, the peptide or fusion protein is produced recombinantly, for example by chemical synthesis.

Also provided are methods of reducing lipoproteins in a sample. In some aspects, the methods include contacting a sample containing lipoproteins with one or more of the disclosed ApoE mimetic peptides under conditions sufficient for binding of lipoproteins to the one or more peptides, thereby forming peptide/lipoprotein complexes and contacting the peptide/lipoprotein complexes with glycosaminoglycans (such as dextran sulfate, heparin, or heparan sulfate) under conditions sufficient for binding of the peptide/lipoprotein complexes to the glycosaminoglycans, thereby forming peptide/lipoprotein/glycosaminoglycan complexes. The methods further include removing the peptide/lipoprotein/glycosaminoglycan complexes from the sample. In some examples, the sample is plasma, such as from a subject with hypercholesteremia.

The foregoing and other features of the disclosure will become more apparent from the following detailed description, which proceeds with reference to the accompanying figures.

### BRIEF DESCRIPTION OF THE DRAWINGS

FIG. 1 is a schematic diagram of a cationic amphipathic alpha-helix formed by the receptor binding region of ApoE (SEQ ID NO: 34).

FIG. 2 is a schematic diagram of an exemplary hydrocarbon stapled ApoE mimetic peptide of a portion of the receptor-binding region of ApoE (SEQ ID NO: 2).

FIG. 3 is a schematic diagram of an exemplary hydrocarbon stapled ApoE mimetic peptide of the receptor-binding region of ApoE (SEQ ID NO: 14).

FIG. 4 is a schematic diagram of an exemplary hydrocarbon stitched ApoE mimetic peptide of the receptor-binding region of ApoE (SEQ ID NO: 15).

FIG. 5 is a schematic diagram of a hydrocarbon stapled ApoE mimetic peptide of amino acids 141-150 of ApoE with N-terminal myristic acid modification (SEQ ID NO: 29).

FIGS. 6A and 6B are graphs showing LDL uptake in HepG2 cells treated with the indicated peptides.

FIGS. 7A and 7B show VLDL uptake (FIG. 7A) and HDL uptake (FIG. 7B) in HepG2 cells treated with the indicated peptides.

FIG. 8 is a graph showing LDL uptake in LDLR-ko HepG2 cells treated with the indicated peptides.

FIGS. 9A-9C show LDL uptake in wild type and LDLR-ko HepG2 cells treated with OctaC-E139-150 (FIG. 9A) or Octa-N-E139-150 (FIG. 9B). FIG. 9C shows LDL uptake in LDLR-KO HepG2 cells mediated by MyrN-E139-150 peptide.

FIGS. 10A and JOB show plasma total cholesterol (FIG. 10A) and triglycerides (FIG. JOB) in mice treated with the indicated peptides. Times are prior to administration (prebleed) or 1, 3, or 6 hours after administration of the peptides. *p<0.01 versus control.

FIG. 11 is a graph showing reduction in mouse hepatitis virus (MHV) GFP fluorescence in HeLa mCC1a cells treated with the indicated ApoE peptides.

FIG. 12 is circular dichroism (CD) spectra of the indicated peptides at 20° C. at 100 μM in water.

FIG. 13 shows solubilization of 1,2,dimyristoyl-sn-glycero-3-phosphocholine (DMPC) phospholipid vesicles by unmodified vs. acylated stapled peptides.

FIGS. 14A and 14B show LDL uptake in the presence of 10 μM peptide (FIG. 14A) and VLDL uptake in the presence of 5 μM peptide (FIG. 14B) by wild type HepG2 cells. Ac-hE18A-NH2 was included as a control (see, e.g., Datta et al., Biochemistry 39:213-220, 2000).

FIG. 15 is a graph showing cell viability in the presence of the indicated peptides and concentrations.

FIG. 16 shows binding of LDL, MyrN-E141-150-amL, or precomplexed LDL/MyrN-E141-150-amL to heparin covered surface of bio-layer interferometry biosensor.

FIG. 17 shows heparin chromatography with LDL or LDL+MyrN-E141-150-amL peptide. NaCl concentration was 0.15 M at baseline and a linear gradient from 0.15 M to 2 M was applied (shown by dashed line).

FIGS. 18A-18C show LDL uptake by heparinase treated wild type HepG2 cells (FIGS. 18A and 18C) or LDLR-KO HepG2 cells (FIG. 18B) mediated by OctaC-E139-150 peptide (FIGS. 18A and 18B) or MyrN-E141-150-amL peptide (FIG. 18C).

FIG. 19 shows the effect of heparin on LDL uptake in wild type HepG2 cells in the presence of MyrN-E141-150-amL peptide.

FIGS. 20A and 20B show effect of MyrN-E139-150 and OctaN-E139-150 peptides on plasma cholesterol (FIG. 20A) and triglycerides (FIG. 20B) in ApoE-KO mice.

FIGS. 21A and 21B show effect of MyrN-E141-150-amL (FIG. 21A) and OctaN-E139-150 (FIG. 21B) peptides on plasma cholesterol in LDLR-KO mice.

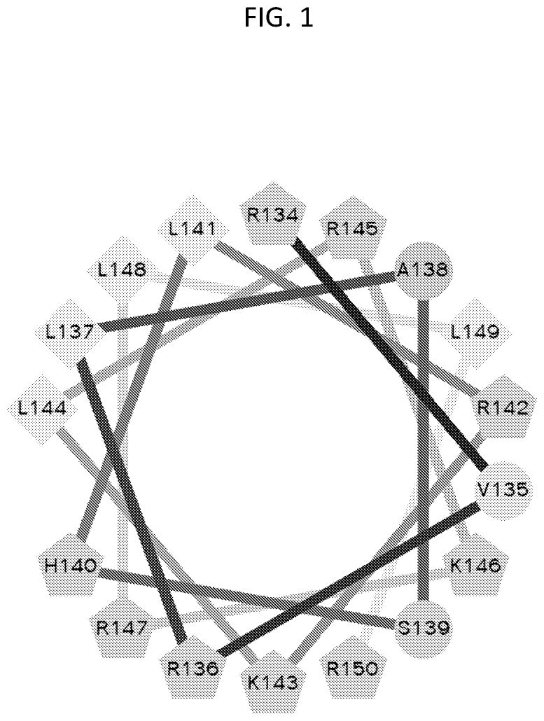

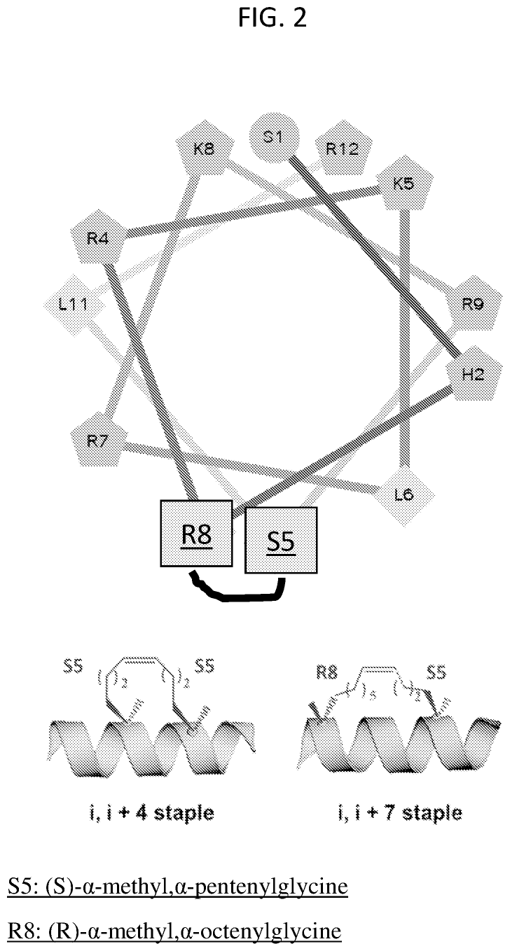

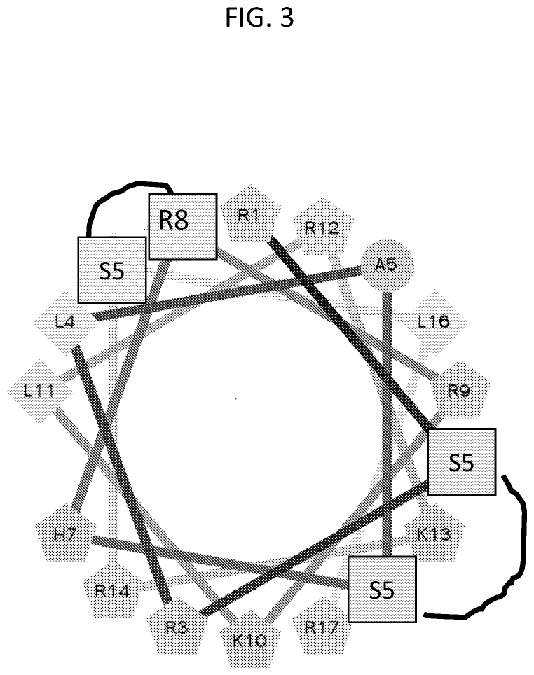

### SEQUENCE LISTING

Any nucleic acid and amino acid sequences provided herein or the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NOs: 1-21 are exemplary ApoE mimetic peptides.

SEQ ID NOs: 22-25 are exemplary ApoE mimetic peptide fusion proteins.

SEQ ID NO: 26 is the amino acid sequence of
an exemplary mature human ApoE protein:
KVEQAVETEPEPELRQQTEWQSGQRWELALGRFWDYLRWVQTLSEQ
VQEELLSSQVTQELRALMDETMKELKAYKSELEEQLTPVAEETRA
RLSKELQAAQARLGADMEDVCGRLVQYRGEVQAMLGQSTEELRVR
LASHLRKLRKRLLRDADDLQKRLAVYQAGAREGAERGLSAIRERL
GPLVEQGRVRAATVGSLAGQPLQERAQAWGERLRARMEEMGSRTR
DRLDEVKEQVAEVRAKLEEQAQQIRLQAEAFQARLKSWFEPLVED
MQRQWAGLVEKVQAAVGTSAAPVPSDNH

SEQ ID NO: 27 is an exemplary human neonatal Fc receptor (FcRn) IgG binding site: RF(penicillarnine)TGHFG(N-methyl-Glycine)(N-methyl-Leucine)YPC-6-aminohexanoic acid

SEQ ID NO: 28 is an exemplary human neonatal Fc receptor (FcRn) albumin binding site: VMHCFWDEEFKCDYG-6-aminohexanoic acid

SEQ ID NO: 29 is an exemplary myristylated ApoE 141-150 peptide.

SEQ ID NOs: 30-33 are additional exemplary ApoE mimetic peptides.

SEQ ID NO: 34 is an exemplary ApoE receptor binding region (amino acids 134-150 of mature human ApoE protein): RVRLASHLRKLRKRLLR

SEQ ID NO: 35 is a portion of an ApoE receptor binding region (amino acids 139-150 of mature human ApoE protein): SHLRKLRKRLLR

SEQ ID NO: 36 is a portion of an ApoE receptor binding region (amino acids 141-150 of mature human ApoE protein): LRKLRKRLLR

SEQ ID NO: 37 is a portion of an ApoE receptor binding region (amino acids 141-148 of mature human ApoE protein): LRKLRKRL

### DETAILED DESCRIPTION

ApoE mimetic peptides are provided herein. The ApoE peptides bind to lipoproteins via the hydrophobic side of the helix (which also contains one or more hydrocarbon staples) and/or acyl chain attached to N- or C-terminus. At the same time, positively charged residues at the hydrophilic side of the helix interact with heparan sulfate proteoglycans (HSPG) and receptors, such as LDLR and possibly LDLR-Related Protein-1 (LRP1).

In some examples, the disclosed peptides provide advantages over previous ApoE mimetics (such as those described in Wolska et al., Cells 10:597, 2021). The advantages include one or more of being shorter in length, more potent, more similar to the native sequence of ApoE, increased resistance to proteolysis, increased plasma half-life, and increased oral bioavailability. As described herein, the disclosed ApoE mimetics decrease plasma total cholesterol and triglycerides in ApoE-ko mice and also decrease plasma total cholesterol in LDLR-ko mice. Thus, they can be used to treat dyslipidemic disorders, such as hypercholesterolemia or hypertriglyceridemia. In addition, the disclosed peptides decrease replication of mouse hepatitis virus, a prototype of betacoronaviruses (such as SARS-CoV-2), demonstrating their usefulness as potential antiviral agents.

### I. Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a peptide” includes singular or plural peptides and can be considered equivalent to the phrase “at least one peptide.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

In order to facilitate review of the various aspects of the disclosure, the following explanations of specific terms are provided:

Alkyl: Refers to a saturated aliphatic hydrocarbyl group having from 1 to 25 (C₁₋₂₅) or more carbon atoms, such as from 1 to 10 (C₁₋₁₀) carbon atoms, from 1 to 4 (C₁₋₄) carbon atoms, from 1 to 14 (C₁₋₁₄) carbon atoms, or from 2 to 22 (C₂₋₂₂) carbon atoms or from 6 to 18 (C₆₋₁₈) carbon atoms. An alkyl moiety may be substituted or unsubstituted. This term includes, by way of example, linear and branched hydrocarbyl groups such as methyl (CH₃), ethyl (—CH₂CH₃), n-propyl (—CH₂CH₂CH₃), isopropyl (—CH(CH₃)₂), n-butyl (—CH₂CH₂CH₂CH₃), isobutyl (—CH₂CH₂(CH₃)₂), sec-butyl (—CH(CH₃)(CH₂CH₃), t-butyl (—C(CH₃)₃), n-pentyl (—CH₂CH₂CH₂CH₂CH₃), neopentyl (—CH₂C(CH₃)₃), hexyl (C₆H₁₃), heptyl (C₇H₁₅), octyl (C₈H₁₇), decyl (C₁₀H₂₁), dodecyl (C₁₂H₂₅), tetradecyl (C₁₄H₂₉), hexadecyl (C₆H₃₃), octadecyl (C₁₈H₃₇) or eicosanyl (C₂₀H₄₁).

Amphipathic: An amphipathic molecule contains both hydrophobic (non-polar) and hydrophilic (polar) groups. One example of an amphipathic molecule is an amphipathic peptide. An amphipathic peptide can also be described as a helical peptide that has hydrophilic amino acid residues on one face of the helix and hydrophobic amino acid residues on the opposite face. In some examples, peptides described herein will form amphipathic helices in vitro or in vivo under appropriate conditions.

Apolipoprotein E (ApoE): A 299 amino acid protein, which plays a role in clearance of triglyceride-rich lipoprotein and cholesterol efflux. There are three different isoforms of ApoE (ApoE2, ApoE3, and ApoE4), which are different at amino acid positions 112 and 158. ApoE2 is associated with type III hyperlipoproteinemia, while ApoE4 is a risk factor for Alzheimer's disease. Mutations in ApoE are associated with familial dysbetalipoproteinemia or type III hyperlipoproteinemia, which feature increased plasma cholesterol and triglycerides, due to impaired clearance of chylomicron and lipoproteins.

Unless the context clearly indicates otherwise, the term ApoE includes any ApoE gene, cDNA, mRNA, or protein from any organism. Nucleic acid and protein sequences for ApoE are publicly available. For example, GenBank Accession No. NM_000041.4 discloses a human ApoE nucleic acid sequence, and GenBank Accession No. NP_000032.1 discloses a human ApoE protein sequence, both of which are incorporated by reference as provided by GenBank on May 5, 2022. In other examples, ApoE includes the mature (processed) form of the protein, for example, lacking the signal sequence. SEQ ID NO: 26 is the amino acid sequence of an exemplary mature human ApoE protein.

Coronavirus: A large family of positive-sense, single-stranded RNA viruses that can infect humans and non-human animals. Coronaviruses get their name from the crown-like spikes on their surface. The viral envelope is comprised of a lipid bilayer containing the viral membrane (M), envelope (E) and spike (S) proteins. Most coronaviruses cause mild to moderate upper respiratory tract illness, such as the common cold.

Betacoronaviruses are a genus of coronaviruses. Three betacoronaviruses have emerged that can cause serious illness and death in humans: severe acute respiratory syndrome coronavirus (SARS-CoV), SARS-CoV-2, and Middle East respiratory syndrome coronavirus (MERS-CoV). Other betacoronaviruses that infect humans include human coronavirus HKU1 (HKU1-CoV) and human coronavirus OC43 (OC43-CoV).

Domain: A domain of a protein is a portion or fragment of a protein that shares common structural, physiochemical and functional features; for example, hydrophobic, polar, globular, helical domains or properties, for example an alpha helical domain, such as an amphipathic helical domain, or a receptor binding domain (such as the receptor binding domain of ApoE).

Dyslipidemic disorder: A disorder associated with an altered amount of any or all of the lipids or lipoproteins in the blood. Dyslipidemic disorders include, for example, hyperlipidemia, hyperlipoproteinemia, hypercholesterolemia, hypertriglyceridemia, HDL deficiency, apoE deficiency, apoA-I deficiency, and cardiovascular disease (e.g., coronary artery disease, atherosclerosis and restenosis).

Familial hypercholesterolemia (FH): A genetic disorder that results in very high plasma LDL levels. FH affects approximately 1 in 200-250 people. Mutations in LDLR, the main receptor responsible for LDL uptake in the liver, are the main cause of FH. Mutations that have an effect on LDLR functionality can cause decreased LDL clearance from circulation. FH patients with extremely high LDL levels may need treatment by LDL-apheresis.

Fused: Linkage by covalent bonding. In some examples, “fused” refers to making two polypeptides into one contiguous polypeptide molecule by recombinant means. For example, a fusion protein is a protein including two separate polypeptides that are linked together.

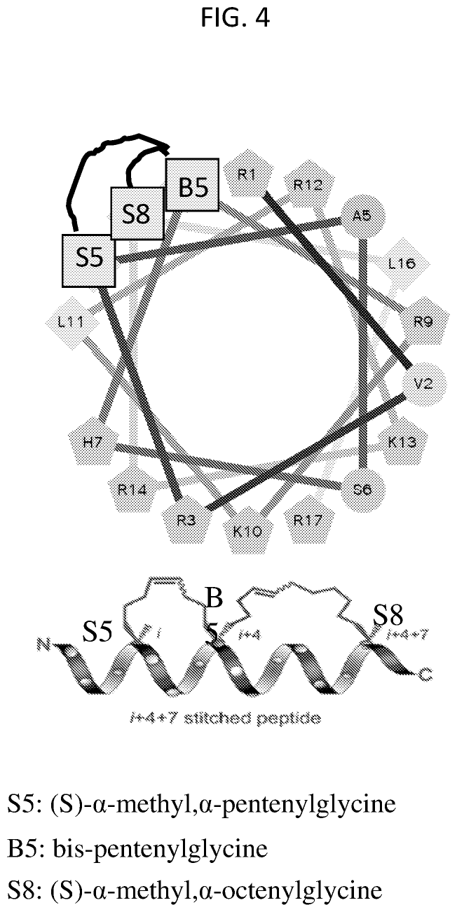

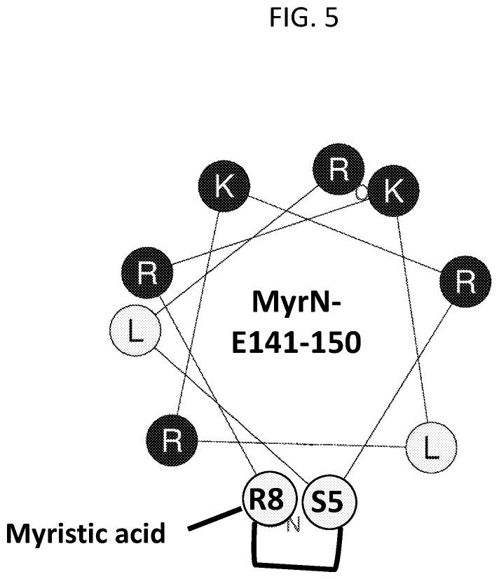

Hydrocarbon Staple or Stitch: A carbon-carbon bond between at least two moieties in a peptide, which in some examples, stabilizes an alpha-helical structure. Hydrocarbon staples are all-hydrocarbon cross-links formed from α,α-disubstituted non-natural amino acids that include terminal olefin tethers. In some examples, the non-natural amino acids are non-contiguous, for example, separated by 3 or 6 amino acids. Hydrocarbon stitching refers to formation of two hydrocarbon staples with a common attachment point at the middle position (e.g., formation of two staples using three non-natural amino acids). Exemplary non-natural amino acids that can be used to form hydrocarbon staples or stitches include (S)-α-methyl,α-pentenylglycine (S5), (S)-α-methyl,α-octenylglycine (S8), (R)-α-methyl,α-pentenylglycine (R5), and (R)-α-methyl,α-octenylglycine (R8), in any combination. In addition, bis-pentenylglycine (B5) can be used to form hydrocarbon stitches, for example, allowing formation of two staples with B5 serving as the common attachment point (see FIG. 4 for an example of a hydrocarbon stitch). Hydrocarbon staples and stitches (which contain adjacent staples) are described in e.g., Walensky and Bird, J. Med. Chem. 57:6275-6288, 2014 and Hilinski et al., J. Am. Chem. Soc. 136:12314-12322, 2014.

Inhibiting or treating a disease: Inhibiting the full development of a disease, disorder or condition, for example, in a subject who is at risk for a disease such as dyslipidemic disorder or a viral infection. “Treatment” refers to a therapeutic intervention that ameliorates a sign or symptom of a disease or pathological condition after it has begun to develop. As used herein, the term “ameliorating,” with reference to a disease, pathological condition or symptom, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters that are specific to the particular disease.

Isolated: An “isolated” biological component (such as a nucleic acid molecule or protein) is one that has been substantially separated or purified away from other biological components in the environment in which the component occurs, e.g., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids or proteins.

Linker or Linkage: A molecule that joins two other molecules, either covalently, or through ionic, van der Waals or hydrogen bonds. In particular examples, a linker comprises a hydrocarbon staple or hydrocarbon stitch. In other examples, a linker is a peptide bond.

Lipoprotein: A biochemical assembly that contains both proteins and lipids, bound to the proteins, which allows fats to move through the water inside and outside cells. There are five major groups of lipoprotein particles, which, in order of molecular size, largest to smallest, are chylomicrons, very low-density lipoprotein (VLDL), intermediate-density lipoprotein (IDL), low-density lipoprotein (LDL), and high-density lipoprotein (HDL). HDL contains the highest proportion of protein to cholesterol; its most abundant apolipoproteins are apoA-I and apoA-II. LDL contains apolipoprotein B, and has a core consisting of linoleate and includes esterified and non-esterified cholesterol molecules. LDL particles are approximately 22 nm in diameter and have a mass of about 3 million Daltons. Lipoprotein a (Lp(a)) is a lipoprotein subclass; lipoprotein a consists of an LDL-like particle and the specific apolipoprotein(a) (apo(a)), which is covalently bound to the apolipoprotein B of the LDL like particle.

Peptide, polypeptide, or protein: A polymer in which the monomers are amino acid residues which are joined together, e.g., through amide bonds. When the amino acids are alpha-amino acids, either the L-optical isomer or the D-optical isomer can be used. The terms “peptide” or “polypeptide” or “protein” as used herein are intended to encompass any amino acid sequence and include modified sequences. The term “peptide” is specifically intended to cover naturally occurring peptides, as well as those which are recombinantly or synthetically produced. “Amino acid” refers to naturally occurring alpha amino acids, unnatural alpha amino acids, natural beta amino acids, and unnatural beta amino acids, as well as modified amino acids (for example, modified by addition of a carbohydrate group, a hydroxyl group, a phosphate group, a fatty acid group, a linker, or a functional group). The term “residue” or “amino acid residue” includes reference to an amino acid that is incorporated into a peptide, polypeptide, or protein.

Unnatural amino acids include without limitation 4-hydroxyproline, desmosine, gamma-aminobutyric acid, beta-cyanoalanine, norvaline, 4-(R)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid. 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines, disubstituted phenylalanines, substituted tyrosines, and statine. Additionally, amino acids may be derivatized to include amino acid residues that are hydroxylated, phosphorylated, sulfonated, acylated, or glycosylated.

In some aspects, the modification involves the substitution of one or more amino acids for amino acids having similar physiochemical and/or structural properties (e.g., “conservative” substitutions). Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the peptide backbone in the area of the substitution, for example, as a helical conformation, (b) the charge or hydrophobicity of the molecule at the target site, or (c) the bulk of the side chain.

In other aspects, one or more substitutions may be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

Pharmaceutically acceptable carriers: The pharmaceutically acceptable carriers useful in this disclosure are known to one of ordinary skill in the art. For example, Remington: The Science and Practice of Pharmacy, Adejare (Ed.), Academic Press, London, United Kingdom, 23^{rd} Edition (2021), describes compositions and formulations suitable for pharmaceutical delivery of the peptides disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Phospholipid: A phospholipid consists of a water-soluble polar head, linked to two water-insoluble non-polar tails (by a negatively charged phosphate group). Both tails consist of a fatty acid, each about 14 to about 24 carbon groups long. When placed in an aqueous environment, phospholipids form a bilayer or micelle, where the hydrophobic tails line up against each other. This forms a membrane with hydrophilic heads on both sides. In some examples, a phospholipid is a lipid that is a primary component of animal cell membranes.

Purified: The term purified does not require absolute purity; rather, it is intended as a relative term. Thus, for example, a purified compound preparation is one in which the compound is more enriched than the compound is in its generative environment, for instance within a cell or in a biochemical reaction chamber. In some aspects, a preparation of compound is purified such that the compound represents at least 50% of the content of the preparation.

Recombinant: A recombinant nucleic acid or protein is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acids, e.g., by genetic engineering techniques. In some examples, a recombinant protein is one encoded for by a recombinant nucleic acid molecule.

Subject: A living multi-cellular vertebrate organism, a category that includes human, veterinary, and laboratory subjects, including human and non-human mammals.

Therapeutically effective amount: A quantity of a specified agent (or combination of agents) sufficient to achieve a desired effect in a subject being treated with that agent, for example, an amount that is sufficient to prevent, treat, reduce, and/or ameliorate the symptoms and/or underlying causes of a disorder or disease. In some aspects, an “effective amount” is an amount that is sufficient to decrease plasma cholesterol and/or triglyceride levels and/or to treat or inhibit a dyslipidemic disorder or viral infection in a subject.

### II. ApoE Mimetic Peptides

Disclosed herein are ApoE mimetic peptides. The peptides include all or a portion of the ApoE receptor-binding region, are 8-17 amino acids long, and include one or more covalent linkages joining at least two non-contiguous amino acids of the peptide. In some aspects, the ApoE mimetic peptides increase cellular lipoprotein uptake, reduce serum triglyceride levels in a subject, reduce total serum cholesterol levels in a subject, or a combination of two or more thereof.

The disclosed ApoE mimetic peptides include the receptor binding region of ApoE (e.g., SEQ ID NO: 34), a modified receptor binding region of ApoE (for example, the amino acid sequence of SEQ ID NO: 34 where one or more amino acids are replaced by a non-natural amino acid), or a portion thereof. In some aspects, the peptides are 8 to 17 amino acids in length (such as 8, 9, 10, 11, 12, 13, 14, 15, 16, or 17 amino acids long). In particular examples, the peptides are 8, 10, 12, or 17 amino acids long. In some aspects, the peptide is 17 amino acids long and includes the amino acids of SEQ ID NO: 34, or a modified version thereof. In other aspects, the peptide is 12 amino acids long and includes SEQ ID NO: 35, or a modified version thereof. In additional aspects, the peptide is 10 amino acids long and includes amino acids of SEQ ID NO: 36, or a modified version thereof. In still further aspects, the peptide is 8 amino acids long and includes amino acids of SEQ ID NO: 37, or a modified version thereof. In any of the disclosed peptides one or more (such as 1, 2, 3, 4, or more) of the amino acids in the sequence may be replaced with a non-natural amino acid that is capable of forming a hydrocarbon staple or stitch.

The peptides include one or more (such as 1, 2, 3, or 4) covalent linkages between two non-contiguous amino acids in the peptide. In some aspects, the peptide includes one covalent linkage between a pair of non-contiguous amino acids in the peptide. In other aspects, the peptide includes two covalent linkages, each covalent linkage being between one of two different pairs of non-contiguous amino acids in the peptide. In still further aspects, the peptide includes two covalent linkages between three non-contiguous amino acids in the peptide, wherein one of the non-contiguous amino acids has a covalent linkage to each of the other two non-contiguous amino acids. In some aspects, the covalent linkage is a hydrocarbon staple, a hydrocarbon stitch, a disulfide bond, or a lactam bridge (e.g., formed by cyclization of lysine ε-amino groups with glutamic or aspartic acid side group carboxyl groups), or other chemical linkages that stabilize helix formation.

When two or more covalent linkages are present, the covalent linkages may be the same or different covalent linkages. In some specific examples, a disclosed peptide includes one hydrocarbon staple, two hydrocarbon staples, one hydrocarbon stitch (comprising two hydrocarbon staples), or one hydrocarbon staple and one lactam bridge. In some examples, the covalent linkage(s) provide one or more improved properties to the peptide, including one or more of stabilization of alpha helical structure, improved binding to lipoproteins, and resistance to proteolysis.

In particular aspects, the disclosed peptides include one or more hydrocarbon staples or hydrocarbon stitches. A hydrocarbon staple is an all-hydrocarbon cross-link formed between two moieties, such as α,α-disubstituted non-natural amino acids that include terminal olefin tethers. A hydrocarbon stitch is made up of two hydrocarbon staples with a common attachment point at a middle position. Thus, in some examples, at least two (such as at least 2, 3, 4, or more) non-contiguous amino acids in the ApoE peptide are replaced with non-natural amino acids that are capable of forming a hydrocarbon staple when linked. In some examples, the non-contiguous amino acids are separated by 3 or 6 amino acids.

Exemplary non-natural amino acids that can be used to form hydrocarbon staples or stitches include (S)-α-methyl,α-pentenylglycine (S5), (S)-α-methyl,α-octenylglycine (S8), (R)-α-methyl,α-pentenylglycine (R5), (R)-α-methyl,α-octenylglycine (R8), bis-pentenylglycine (B5), (S)-pentenylalanine. (S)-octenylalanine, (R)-pentenylalanine, and (R)-octenylalanine, in any combination. In some examples, the non-natural amino acids are selected such that they are capable of forming a hydrocarbon staple. Thus, for example, if the two non-natural amino acids are separated by 2 or 3 amino acids, the hydrocarbon staple is “short” and the linkage is between two pentenyl-modified amino acids (such as between S5 and S5, between R5 and R5, between S5 and R5, or between R5 and S5). If the two non-natural amino acids are separated by 6 amino acids, the hydrocarbon staple is “long” and the linkage is between a pentyl-modified amino acid and an octenyl-modified amino acid (such as between R8 and S5 or between S8 and R5). In other examples, the non-natural amino acids are selected such that they are capable of forming a hydrocarbon stitch. In one specific example a hydrocarbon stitch is formed by including an octenyl-modified amino acid separated from a bis-pentenylglycine residue by 6 amino acids, which in turn is separated from a pentenyl-modified amino acid by 3 amino acids (such as R8-B5-R5, R8-B5-S5, S8-B5-S5, or S8-B5-R5). In other examples, a hydrocarbon stitch is formed by including a pentynyl-modified amino acid separated from a bis-pentenylglycine residue by 3 amino acids, which in turn is separated from an octenyl-modified amino acid by 6 amino acids (such as S5-B5-S8, S5-B5-R8, R5-B5-R8, or R5-B5-S8). In another example, a hydrocarbon stitch is formed by including an octenyl-modified amino acid separated from a bis-pentenylglycine residue by 6 amino acids, which in turn is separated from a second octenyl-modified amino acid by 6 amino acids (such as R8-B5-S8, S8-B5-R8, R8-B5-R8, or S8-B5-S8). In yet another example, a hydrocarbon stitch is formed by including a pentynyl-modified amino acid separated from a bis-pentenylglycine residue by 3 amino acids, which in turn is separated from an pentynyl-modified amino acid by 3 amino acids (such as R5-B5-S5, R5-B5-R5, S5-B5-R5, or S5-B5-S5).

Also encompassed by the present disclosure are peptides including one or more additional modifications in addition to the covalent linkages discussed above and any modifications required to create such linkages. In some aspects the peptide includes one or more modifications including addition, deletion, and/or substitution of one or more amino acids in the ApoE mimetic peptides with another naturally occurring amino acid residue, for example, compared with the naturally occurring ApoE amino acid sequence, or with a non-naturally occurring amino acid. In some examples, a leucine (such as a leucine corresponding to leucine 149 of SEQ ID NO: 26) may be replaced with alpha-methyl-leucine, N-methyl-leucine, or β-Leucine. In other examples, an arginine (such as an arginine corresponding to arginine 150 of SEQ ID NO: 26) may be replaced with homoarginine. In further examples, one or more lysine residues in the peptide may be replaced with arginine or homoarginine. In some examples, the substitution(s) may make the peptides more resistant to proteolysis, increase plasma half-life, increase bioavailability, or a combination of two or more thereof.

In addition to naturally occurring genetically encoded amino acids, one or more amino acid residues in the disclosed peptides may be substituted with naturally occurring non-encoded amino acids and/or synthetic amino acids. In some aspects, the substitution(s) may increase lipid binding, increase resistance to proteolysis, increase oral bioavailability, or a combination thereof. In some examples, such amino acids include acylation of lysine ε-amino groups; N-alkylation of arginine, histidine, or lysine; alkylation of glutamic or aspartic carboxylic acid groups; deamidation of glutamine or asparagine; β-alanine and other omega-amino acids, such as 3-aminopropionic acid, 2,3-diaminopropionic acid, 4-aminobutyric acid and the like; α-aminoisobutyric acid; ε-aminohexanoic acid; δ-aminovaleric acid; N-methylglycine or sarcosine; ornithine; citrulline; penicillamine; t-butylalanine; t-butylglycine; N-methylisoleucine; phenylglycine; cyclohexylalanine; norleucine; naphthylalanine; 4-chlorophenylalanine; 2-fluorophenylalanine; 3-fluorophenylalanine; 4-fluorophenylalanine; alpha-methyl-leucine; N-methyl-leucine; β-Leucine; 1,2,3,4-tetrahydroisoquinoline-3-carboxylic acid; β-2-thienylalanine; methionine sulfoxide; homoarginine; N-acetyl lysine; alpha-methyl-lysine; 2,4-diaminobutyric acid; 2,3-diaminobutyric acid; p-aminophenylalanine; N-methyl valine; homocysteine; homophenylalanine; homoserine; hydroxyproline; homoproline; N-methylated amino acids; peptoids (where side groups are appended to the nitrogen atom of the peptide backbone, rather than to the α-carbons); and β-peptides (where the amino group is bonded to the β carbon rather than the α-carbon). In one specific example, one or more leucine residues in the peptide (such as the leucine corresponding to amino acid 149 of SEQ ID NO: 26) is replaced with alpha-methyl-leucine or N-methyl-leucine.

While in certain aspects, the amino acids of the disclosed ApoE mimetic peptides are L-amino acids, in other aspects, one or more (such as 1, 2, 3, 4, 5, 6, 7, 8, 9, 10 or more, or all) L-amino acids are replaced with a D-amino acid (e.g., L-Leu→D-Leu). In some examples, including one or more D-amino acids may make the peptide more resistant to proteolysis, increase plasma half-life, and/or increase oral bioavailability. In some aspects, all amino acids in the peptide are D-amino acids. In other aspects, one or more amino acids (such as 1, 2, 3, 4, 5, 6, 7, 8, 9, 10, 11, 12, or more) are D-amino acids. In some examples, L-Lys is changed to D-Arg or D-homoarginine or L-Arg is changed to D-Lys or D-homoarginine. In some examples, at least 1, 2, or 3 C-terminal amino acids are D-amino acids. Specific, non-limiting examples include the peptides of any one of SEQ ID NOs: 7-10, 32, and 33. In additional examples, a modified or non-naturally occurring amino acid, non-encoded amino acid, or synthetic amino acid (such as those described above) may be a D-amino acid.

In further aspects, the disclosed peptides may include an N-terminal modification, a C-terminal modification, or both. In some examples, the peptide includes N-terminal acylation, C-terminal amidation, or both. In other examples, the modification includes an N-terminal or C-terminal fatty acid, for example, to increase binding of the peptide to lipoproteins. In some examples, the fatty acid is a C6-C18 fatty acid (from hexanoic acid to stearic acid). In specific examples, the fatty acid is octanoic acid (also known as caprylic acid) or myristic acid.

In other examples, N-terminal modifications include the desamino, N-lower alkyl, N-di-lower alkyl, constrained alkyls (e.g. branched, cyclic, fused, adamantyl), and N-acyl modifications. Exemplary C-terminal modifications include the amide, lower alkyl amide, constrained alkyls (e.g. branched, cyclic, fused, adamantyl) alkyl, dialkyl amide, and lower alkyl ester modifications. Lower alkyl is C1-C4 alkyl.

In some aspects, the disclosed ApoE mimetic peptides include an amino acid sequence with at least 90% sequence identity (such as at least 90%, at least 91%, at least 92%, at least 93%, at least 94%, at least 95%, at least 96%, at least 97%, at least 98%, or at least 99% sequence identity) to the amino acid sequence of any one of SEQ ID NOs: 2-4, 7-12, 14-21, and 30-33. In other examples, the amino acid sequence of the ApoE mimetic peptides include or consist of the amino acid sequence of any one of SEQ ID NOs: 2-4, 7-12, 14-21, and 30-33. Exemplary hydrocarbon stapled or stitched ApoE mimetic peptides are provided in Table 1.

TABLE 1
Exemplary ApoE peptides

| | | SEQ ID |
|---|---|---|
| Peptide Name | Sequence | NO: |
| E139-150 (control) | Ac-SHLRKLRKRLLR-NH2 | 1 |
| E139-150-S | Ac-SH(R8)RKLRKR(S5)LR-NH2 | 2 |
| OctaN-E139-150 (also | Octanoyl-SH(R8)RKLRKR(S5)LR-NH2 | 3 |
| referred to as OctaN- | | |
| E139-150-S) | | |
| OctaC-E139-150 (also | Ac-SH(R8)RKLRKR(S5)LR-Octanoyl | 4 |
| referred to as OctaC- | | |
| E139-150-S) | | |
| OctaN-NS-E139-150 | Octanoyl-SHLRKLRKRLLR-NH2 | 5 |
| (control) | | |
| OctaC-NS-E139-150 | Ac-SHLRKLRKRLLR-Octanoyl | 6 |
| (control) | | |
| D-OctaC-E139-150 | Ac-sh(S8)rklrkr(R5)lr-Octanoyl | 7 |
| D-OctaN-E139-150 | Octanoyl-sh(S8)rklrkr(R5)lr-NH | 8 |
| D-Ir-OctaC-E139-150 | Ac-SH(R8)RKLRKR(S5)lr-Octanoyl | 9 |
| D-Ir-OctaN-E139-150 | Octanoyl-SH(R8)RKLRKR(S5)lr-NH2 | 10 |
| MyrC-E139-150 | Ac-SH(R8)RKLRKR(S5)LR-Myristyl | 11 |
| MyrN-E139-150 | Myristyl-SH(R8)RKLRKR(S5)LR-NH2 | 12 |
| E134-150 (control) | Ac-RVRLASHLRKLRKRLLR-NH2 | 13 |
| E134-150-SS | Ac-R(S5)RLA(S5)H(R8)RKLRKR(S5)LR-NH2 | 14 |
| E134-150-Stitch | Ac-RVR(S5)ASH(B5)RKLRKR(S8)LR-NH2 | 15 |
| OctaN-E141-150 | Octanoyl-(R8)RKLRKR(S5)LR-NH2 | 16 |
| OctaC-E141-150 | Ac-(R8)RKLRKR(S5)LR-Octanoyl | 17 |
| OctaN-E139-148 | Octanoyl-SH(R8)RKLRKR(S5)-NH2 | 18 |
| OctaC-E139-148 | Ac-SH(R8)RKLRKR(S5)-Octanoyl | 19 |
| OctaN-E141-148 | Octanoyl-(R8)RKLRKR(S5)-NH2 | 20 |
| OctaC-E141-148 | Ac-(R8)RKLRKR(S5)-Octanoyl | 21 |
| MyrN-NS-E141-150 | Myristyl-LRKLRKRLLR-NH2 | 29 |
| (control) | | |
| MyrN-E141-150 | Myristyl-(R8)RKLRKR(S5)LR-NH2 | 30 |
| MyrN-E141-150-amL | Myristyl-(R8)RKLRKR(S5)L | 31 |
| | (alpha-methyl)R-NH2 | |
| D-OctaN-E141-150 | Octanoyl-(S8)rklrkr(R5)lr-NH2 | 32 |
| D-MyrN-E141-150 | Myristyl-(S8)rklrkr(R5)lr-NH2 | 33 |

Ac: N-terminal acylation
NH2: C-terminal amidation
S5: (S)-α-methyl,α-pentenylglycine
R8: (R)-α-methyl,α-octenylglycine
S8: (S)-α-methyl,α-octenylglycine
R5: (R)-α-methyl,α-pentenylglycine
B5: bis-pentenylglycine
Lower case indicates D-amino acids

Also provided are fusion proteins that include a disclosed ApoE mimetic peptide linked or fused to an additional peptide. In some aspects, the ApoE mimetic peptide is linked to a peptide or modified peptide that can improve oral bioavailability and/or increase plasma half-life of the peptide. The additional peptide may be fused to the N-terminus of the ApoE mimetic peptide or to the C-terminus of the ApoE mimetic peptide. In some aspects, the ApoE mimetic peptide and the additional peptide are contiguous (e.g., directly linked). In other aspects, the ApoE mimetic peptide and the additional peptide are connected via a linker, such as a peptide linker. In some examples, the peptide linker is 1, 2, 3, 4, or more amino acids long), for example 1-3 glycine residues or a proline residue. In other examples, the linker is a bifunctional linker (such as succinimide), a disulfide bond, 6-aminohexanoic acid, or a non-coded amino acid, such as isoaminobutyric acid.

IgG and albumin have exceptionally long plasma half-lives, whereas most other human proteins exhibit rapid blood clearance. This long half-life of IgG and albumin is a result of interaction with neonatal Fc receptor (FcRn), which creates an intracellular protein reservoir that is protected from lysosomal degradation and subsequently recycled to the extracellular space. Genetic fusion between a therapeutic protein and either the Fc domain of IgG, albumin, or long, flexible polypeptide extensions has been introduced to extend protein circulation. FcRn can bind IgG at pH 6 but not bind at physiological pH (7.4), and this pH dependence is likely key to the mechanism by which FcRn extends IgG half-lives. It is thought that after uptake of IgG into cells, FcRn can bind to IgG in acidic endosomes, thereby avoiding degradation in the lysosome. IgG molecules are then returned to the cell surface by exocytosis and released back into circulation because FcRn has minimal affinity for IgG at extracellular pH 7.4.

In some examples, the ApoE mimetic peptide is linked to a human neonatal Fc receptor (FcRn) binding sequence, such as an IgG binding site (e.g., SEQ ID NO: 27), an albumin binding site (e.g., SEQ ID NO: 28), or a non-IgG/non-albumin FcRn binding site. See, e.g., U.S. Pat. Nos. 9,527,890; 9,012,603; 9,574,190; 10,046,023; 10,316,073; 10,588,935; Mezo et al., Proc. Natl. Acad. Sci. USA 105:2337-2342, 2008.

The fusion proteins can include any of the modifications discussed above with respect to the ApoE mimetic peptides. In particular examples, the fusion proteins include one or more of N-terminal acylation, C-terminal amidation, N-terminal fatty acid modification (such as octanoic acid or myristic acid), and C-terminal fatty acid modification (such as octanoic acid or myristic acid).

In some aspects, the fusion protein includes an amino acid sequence with at least 90% sequence identity (such as at least 90%, at least 91%, at least 92%, at least 93%, at least 94%, at least 95%, at least 96%, at least 97%, at least 98%, or at least 99% sequence identity) to the amino acid sequence of any one of SEQ ID NOs: 22-25. In other examples, the amino acid sequence of the fusion protein includes or consists of the amino acid sequence of any one of SEQ ID NOs: 22-25. Exemplary fusion proteins include those provided in Table 2.

TABLE 2
Exemplary ApoE mimetic peptide fusion proteins

| | | SEQ |
|---|---|---|
| | | ID |
| Peptide Name | Sequence | NO: |
| IgG/FcRn-OctaC- | Ac-RF1TGHFG23YPC4SH(R8) | 22 |
| E139-150 | RKLRKR(S5)LR-octanoyl | |
| IgG/FcRn-OctaN- | Octanoyl-SH(R8)RKLRKR | 23 |
| E139-150 | (S5)LR4RF1TGHFG23YPC | |
| Albumin/FcRn- | Ac-VMHCFWDEEFKCDYG4SH | 24 |
| OctaC-E139-150 | (R8)RKLRKR(S5)LR- | |
| | octanoyl | |
| Albumin/FcRn- | Octanoyl- | 25 |
| OctaN-E139-150 | SH(R8)RKLRKR(S5)LR4V | |
| | MHCFWDEEFKCDYG-NH₂ | |

1: Penicillamine
2: N-methyl-Glycine
3: N-methyl-Leucine
4: 6-aminohexanoic acid
Ac: N-terminal acylation
NH2: C-terminal amidation
S5: (S)-α-methyl,α-pentenylglycine
R8: (R)-α-methyl,α-octenylglycine

In some aspects, the disclosed ApoE mimetic peptides or fusion proteins can be isolated from various sources. For example, a disclosed peptide or fusion protein can be produced by chemical synthesis (such as solid-phase or solution-phase synthesis), followed by reaction under appropriate conditions to create the one or more covalent linkages between two or more non-contiguous amino acids of the peptides. In some examples, the covalent linkage is a hydrocarbon staple or hydrocarbon stitch and the hydrocarbon staple or hydrocarbon stitch is produced by ruthenium-catalyzed metathesis or bis ring-closing metathesis.

In some aspects, the ApoE mimetic peptides or fusion proteins disclosed herein retain one or more activities of ApoE, including lipoprotein binding activity, LDLR binding activity, LRP1 binding activity, heparan sulfate proteoglycan (HSPG) binding activity, mediating cellular lipoprotein uptake, reducing serum triglyceride levels in a subject, reducing total serum cholesterol levels in a subject, and/or reducing viral replication in a cell or in a subject. In some examples, the disclosed peptides or fusion proteins increase cellular lipoprotein uptake (such as LDL, IDL, HDL, Lp(a), chylomicron, chylomicron remnant, or VLDL uptake) by at least about 5- to 40-fold compared to a control (such as a corresponding peptide with native ApoE sequence that does not include covalent linkages or no peptide/buffer). In further examples, the disclosed peptides or fusion proteins decrease serum triglycerides by at least about 50% (such as at least about 50%, at least about 55%, at least about 60%, at least about 65%, at least about 70%, at least about 75%, at least about 80%, at least about 85%, at least about 90%, at least about 95%, or more) in one hour compared to a control (such as a corresponding peptide with native ApoE sequence that does not include covalent linkages or no peptide/buffer) when administered to ApoE-ko mice. In additional examples, the disclosed peptides or fusion proteins decrease serum cholesterol by at least about 10% (for example, at least about 10%, at least about 15%, at least about 20%, at least about 25%, or more) in 1-6 hours compared to a control (such as a corresponding peptide with native ApoE sequence that does not include covalent linkages or no peptide/buffer) when administered to ApoE-ko mice. In further examples, the disclosed peptides or fusion proteins decrease viral replication in cells by at least about 50% compared to a control (such as a corresponding peptide with native ApoE sequence that does not include covalent linkages or no peptide/buffer).

### III. Pharmaceutical Compositions and Methods of Use

In exemplary aspects, compositions including one or more of the ApoE mimetic peptides or fusion proteins disclosed herein are administered to a subject suffering from a dyslipidemic disorder, such as hypertriglyceridemia, hypercholesterolemia, or cardiovascular disease, in a therapeutically effective amount. In other examples, a therapeutically effective amount of ApoE mimetic peptides or fusion proteins disclosed herein are administered to a subject with a genetic deficiency of ApoE or a subject with dysbetalipoproteinemia (such as a subject with ApoE2/E2 alleles). In additional examples, a therapeutically effective amount of ApoE mimetic peptides or fusion proteins are administered to a subject with familial hypercholesterolemia due to mutations in LDLR. In other aspects, a therapeutically effective amount of a composition including one or more of the disclosed ApoE mimetic peptides or fusion proteins disclosed herein are administered to a subject with a viral infection or disease. Amounts effective for these uses will depend upon the severity of the disorder and the general state of the subject's health, among other factors. A therapeutically effective amount of the compound is that which provides either subjective relief of one or more symptoms or an objectively identifiable improvement as noted by a clinician or other qualified observer.

An ApoE mimetic peptide or fusion protein can be administered by any means known to one of skill in the art (see, e.g., Banga, Therapeutic Peptides and Proteins, Third Edition, Taylor & Francis Group, 2015), such as by intramuscular, subcutaneous, or intravenous injection, or oral, nasal, inhalation, or anal administration. In one aspect, administration is by intravenous injection. In another aspect, administration is oral. To extend the time during which the ApoE mimetic peptide or fusion protein is available to inhibit or treat a dyslipidemic disorder or a viral infection, the peptide can be provided as an implant, an oily injection, or as a particulate system. The particulate system can be a microparticle, a microcapsule, a microsphere, a nanocapsule, or similar particle. In specific aspects, the ApoE mimetic peptide that is administered includes the amino acid sequence of any one of SEQ ID NOs: 2-4, 7-12, 14-21, and 30-33. In other aspects, the subject is administered a fusion protein including an ApoE mimetic peptide, such as any one of SEQ ID NOs: 22-25.

In some examples, the methods include administering a therapeutically effective amount of a disclosed ApoE mimetic peptide or fusion protein to a subject in need thereof. In some examples, the subject is a human or veterinary subject. In particular examples, the subject is human.

In some examples, the subject has hypertriglyceridemia. Normal or desirable serum triglycerides in a human subject is less than 150 mg/dL. The borderline high triglyceride range is 150-199 mg/dL, high triglyceride range is 200-499 mg/dL, and very high triglyceride range is 500 mg/dL or more. Thus, in some examples, the subject being treated has serum triglycerides of 150 mg/dL or more. The subject is administered a therapeutically effective amount of one or more of the disclosed peptides or fusion proteins, such as an amount that results in a decrease in serum triglyceride levels by about at least 5% (such as at least about 5%, at least about 10%, at least about 15%, at least about 20%, at least about 25%, at least about 30%, at least about 40%, at least about 50% or more), for example, compared to triglyceride levels prior to administration of the peptide or fusion protein. In other examples, a therapeutically effective amount of the disclosed peptides or fusion proteins reduces serum triglycerides in the subject to less than 150 mg/dL.

In some examples, the subject has hypercholesterolemia, including but not limited to familial hypercholesterolemia. Normal or desirable total cholesterol in an adult human subject is less than 200 mg/dL (less than 170 mg/dL for children). The borderline high total cholesterol range for adults is 200-239 mg/dL, and the high total cholesterol range is 240 mg/dL or more (170-199 mg/dL and 200 mg/dL or more, respectively, in children). Thus, in some examples, the subject being treated is an adult and has a serum total cholesterol level of 200 mg/dL or more. The subject is administered a therapeutically effective amount of one or more of the disclosed peptides or fusion proteins, such as an amount that results in a decrease in serum total cholesterol levels by about at least 5% (such as at least about 5%, at least about 10%, at least about 15%, at least about 20%, at least about 25%, at least about 30%, at least about 40%, at least about 50% or more), for example, compared to total cholesterol levels prior to administration of the peptide or fusion protein. In other examples, a therapeutically effective amount of the disclosed peptides or fusion proteins reduces serum total cholesterol in the subject to less than 200 mg/dL.

In other aspects, the subject has or is suspected to have a viral infection or disease. In some examples, the subject is infected with or is suspected to be infected with an enveloped virus. Exemplary enveloped viruses include influenza viruses, herpesviruses (such as cytomegalovirus), poxviruses, hepadnaviruses (such as hepatitis B virus), flaviviruses (such as Dengue virus or West Nile virus), coronaviruses (such as betacoronaviruses), orthomyxoviruses, paramyxoviruses (such as measles or mumps), filoviruses (such as Ebola virus or Marburg virus), and retroviruses (such as HIV). In some examples, the subject is infected with (or is suspected to be infected with) a betacoronavirus, for example, SARS-CoV-2. In one aspect, the subject is administered a therapeutically effective amount of the peptide, such as an amount necessary to inhibit viral replication by at least 10%, at least 20%, at least 30%, at least 40%, at least 50%, at least 60%, at least 70%, at least 80%, or at least 90% (as compared to the absence of treatment). In another aspect, a therapeutically effective amount is the amount necessary to reduce viral titer in a subject by at least 10%, at least 20%, at least 30%, at least 40%, at least 50%, at least 60%, at least 70%, at least 80%, or at least 90% (as compared to the absence of treatment). In other examples, the therapeutically effective amount can also be the amount necessary to reduce or eliminate one of more symptoms of viral infection, such as the amount necessary reduce or eliminate fever, cough, or shortness of breath, for example in the case of a respiratory virus, such as a betacoronavirus.

One or more than one of the provided ApoE mimetic peptides or fusion proteins can be combined with one or more pharmaceutically acceptable carriers or excipients for administration to human or animal subjects. Remington: The Science and Practice of Pharmacy, Adejare (Ed.), Academic Press, London, United Kingdom, 23^{rd} Edition (2021), describes compositions and formulations suitable for pharmaceutical delivery of one or more therapeutic compounds or molecules, such as one or more peptides or fusion proteins alone or in combination with additional pharmaceutical agents. Examples of suitable pharmaceutically acceptable carriers, vehicles, or excipients include sterile aqueous or non-aqueous solutions, suspensions, and/or emulsions. Examples of non-aqueous solvents include propylene glycol, polyethylene glycol, vegetable oils such as olive oil, and injectable organic esters such as ethyl oleate. Aqueous carriers include water, alcoholic/aqueous solutions, emulsions, or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Additional pharmaceutically acceptable carriers include carbohydrates (for example, glucose, sucrose, or dextrans), antioxidants (such as ascorbic acid or glutathione), chelating agents, low molecular weight proteins, lipids, wetting agents, emulsifying agents, dispersing agents, and preservatives.

In general, the formulations are prepared by uniformly and intimately bringing into association one or more of the disclosed peptides or fusion proteins with the pharmaceutically acceptable carrier(s) or excipient(s). Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of a sterile liquid carrier, for example, water or saline for injections, immediately prior to use. Extemporaneous injection solutions and suspensions may be prepared from sterile powders, granules and tablets commonly used by one of ordinary skill in the art.

In some aspects, the disclosed peptides or fusion proteins are formulated as a complex with one or more phospholipids. In one aspect, one or more of the disclosed peptides or fusion proteins are precomplexed with phospholipids or other lipids into either discoidal or spherical shape particles prior to administration to subjects. In one example, the peptides are precomplexed with 1, 2-dimyristoyl-sn-glycero-3-phosphocholine (DMPC). Methods of preparing complexes of the disclosed peptides with phospholipids include those described in Schwendeman et al., J. Lipid Res. 56:1727-1737, 2015; Yuan et al., Nanomedicine: Nanotechnology, Biology and Medicine 48:102646, 2023; and Shu et al., Aterioscler Thromb Vasc Biol. 30:2504-2509, 2010.

The peptides, fusion proteins, and pharmaceutical compositions provided herein, including those for use in treating dyslipidemic disorders or viral diseases, may be administered through different routes, such as oral, including buccal and sublingual, rectal, parenteral (including intravenous or intraperitoneal), aerosol, nasal, intramuscular, subcutaneous, intradermal, and topical. They may be administered in different forms, including but not limited to solutions, emulsions and suspensions, microspheres, particles, microparticles, nanoparticles, and liposomes.

The amount of the peptide, fusion protein, or pharmaceutical composition that will be effective depends on the nature of the disorder or condition to be treated, as well as the stage of the disorder or condition. Effective amounts can be determined by standard clinical techniques. The precise dose to be employed in the formulation will also depend on the route of administration, and should be decided according to the judgment of the health care practitioner and each subject's circumstances. An example of such a dosage range is about 0.1 mg/kg to about 200 mg/kg body weight (for example, about 0.1 to about 10 mg/kg, about 1 to about 25 mg/kg, about 5 to about 50 mg/kg, about 25 to about 75 mg/kg, about 50 to about 100 mg/kg, about 75 to about 150 mg/kg, or about 100 to about 200 mg/kg, such as about 0.1 mg/kg, about 0.5 mg/kg, about 1 mg/kg, about 2.5 mg/kg, about 5 mg/kg, about 7.5 mg/kg, about 10 mg/kg, about 15 mg/kg, about 20 mg/kg, about 25 mg/kg, about 50 mg/kg, about 75 mg/kg, about 100 mg/kg, about 125 mg/kg, about 150 mg/kg, about 175 mg/kg, or about 200 mg/kg) in single or divided doses. Another example of a dosage range is 1.0 to 100 mg/kg body weight in single or divided doses. In some aspects, the disclosed peptides, fusion proteins, or pharmaceutical compositions are administered twice per day, daily, every other day, weekly, or less frequently. In one specific example, the subject is administered the peptide, fusion protein, or composition intravenously once weekly. In another specific example, the subject is administered the peptide, fusion protein, or composition orally once per day. Treatment may continue indefinitely, or may be discontinued when a target clinical parameter is reached, such as normal range triglyceride or total cholesterol levels or clearance of a viral infection (for example, resolution of symptoms or lack of viremia).

The specific dose level and frequency of dosage for any particular subject may be varied and will depend upon a number of factors, including the particular disorder or condition, the activity of the specific compound, the metabolic stability and length of action of that compound, the age, body weight, general health, sex, diet, mode and time of administration, rate of excretion, drug combination, and severity of the condition of the subject undergoing therapy.

The disclosed peptides, fusion proteins, or pharmaceutical compositions can be administered at about the same dose throughout a treatment period, in an escalating dose regimen, or in a loading-dose regime (e.g., in which the loading dose is about two to five times the maintenance dose). In some aspects, the dose is varied during the course of a treatment based on the condition of the subject being treated, the severity of the disease or condition, the apparent response to the therapy, and/or other factors as judged by one of ordinary skill in the art.

In additional aspects, the disclosed peptides are utilized in in vitro or ex vivo methods to facilitate or increase removal of VLDL, LDL, Lp(a), and/or Lipoprotein X (LpX) from a sample, such as plasma. In some examples, the disclosed peptides are utilized in methods of LDL-apheresis (such as for subjects with familial hypercholesterolemia). In some aspects, the methods include contacting a sample containing lipoproteins (such as a biological sample from a subject, for example, plasma) with one or more of the disclosed peptides under conditions sufficient for binding of lipoproteins (such as LDL and/or VLDL) to the one or more peptides, thereby forming peptide/lipoprotein complexes. The peptide/lipoprotein complexes are then contacted with glycosaminoglycans (for example, dextran sulfate, heparin, or heparan sulfate) under conditions sufficient for binding of the peptide/lipoprotein complexes to the glycosaminoglycans, thereby forming peptide/lipoprotein/glycosaminoglycan complexes. In one specific example, the glycosaminoglycan is dextran sulfate. The peptide/lipoprotein/glycosaminoglycan complexes are removed from the sample, resulting in lipoprotein-depleted plasma. In some aspects, the glycosaminoglycan is immobilized on a solid support (such as a column) or is in the form of a slurry. In some examples, the methods reduce the amount of lipoproteins (such as LDL) in the sample by at least about 30% (such as at least about 30%, at least about 40%, at least about 50%, at least about 60%, at least about 70%, at least about 80%, or more). In additional examples, inclusion of a disclosed peptide or fusion protein increases the amount of lipoproteins (such as LDL) removed from the sample compared to use of glycosaminoglycans alone.

In some examples, the method can be used in methods of removing lipoproteins (such as LDL) from plasma of a subject (such as a subject with familial hypercholesterolemia) by LDL-apheresis. LDL-apheresis systems include cascade filtration (or lipid filtration), immunoadsorption, heparin-induced LDL precipitation, dextran sulfate LDL adsorption, and LDL hemoperfusion. In some aspects, the methods utilize heparin-induced LDL precipitation apheresis. In some examples, blood is removed from the subject and plasma is separated from blood cells utilizing a blood processing system (for example, a Liposorber® system; Kaneka Pharma America LLC). The separated plasma is contacted with one or more disclosed peptides or fusion proteins under conditions sufficient for binding of lipoproteins (such as LDL and/or VLDL) to the one or more peptides or fusion proteins, thereby forming peptide/lipoprotein complexes. The plasma containing the peptide/lipoprotein complexes is then flowed through a column containing dextran sulfate chains. The peptide/lipoprotein complexes are retained on the column and the amount of lipoprotein in the plasma is reduced. Blood cells are added back to the plasma and the blood is returned to the subject.

### EXAMPLES

The following examples are provided to illustrate particular features of aspects of the disclosure. These examples should not be construed to limit the disclosure to the particular features described.

### Example 1

### In Vitro Testing of ApoE Mimetic Peptides

Lipid uptake in wild type and LDLR-ko HepG2 cells was assessed as described in Lucero et al. (J. Lipid Res. 63(1), Article 100160, 2022). Briefly, HepG2 cells or HepG2 LDLR-ko cells were seeded on a 96-well plate 48 hours before the experiment (25,000 cells per well) in media containing DMEM, 10% (v/v) FBS, 100 IU/ml penicillin G, and 100 μg/ml streptomycin. Proteins in LDL, HDL, or VLDL were labeled with amino-reactive fluorescent dye Alexa Fluor™ 568 NHS Ester (Succinimidyl Ester) (Invitrogen). Alexa568-LDL (or -HDL or -VLDL) at 50 μg/ml final total protein concentration was mixed with different apoE peptides at various concentrations or with same amount of buffer in DMEM/0.1% BSA. Alexa568-lipoprotein/peptide mixtures or Alexa568-lipoprotein was then preincubated at 37° C. for 30 min. Cells were washed with PBS and Alexa568-lipoprotein/peptide mixtures or Alexa568-lipoprotein in DMEM/0.1% BSA were added to the cells on 96-well plates (in triplicates) and incubated for 1 h or 4 h. Cells were washed with PBS, dissociated from wells with trypsin, and resuspended in ice cold PBS/0.5% BSA/2.5 mM EDTA. Uptake of LDL/HDL/VLDL by cells was assessed by measuring fluorescence with fluorescence-activated cell sorting (FACS) flow cytometry (BD LRSFortessa (BD Biosciences, Franklin Lakes, NJ)).

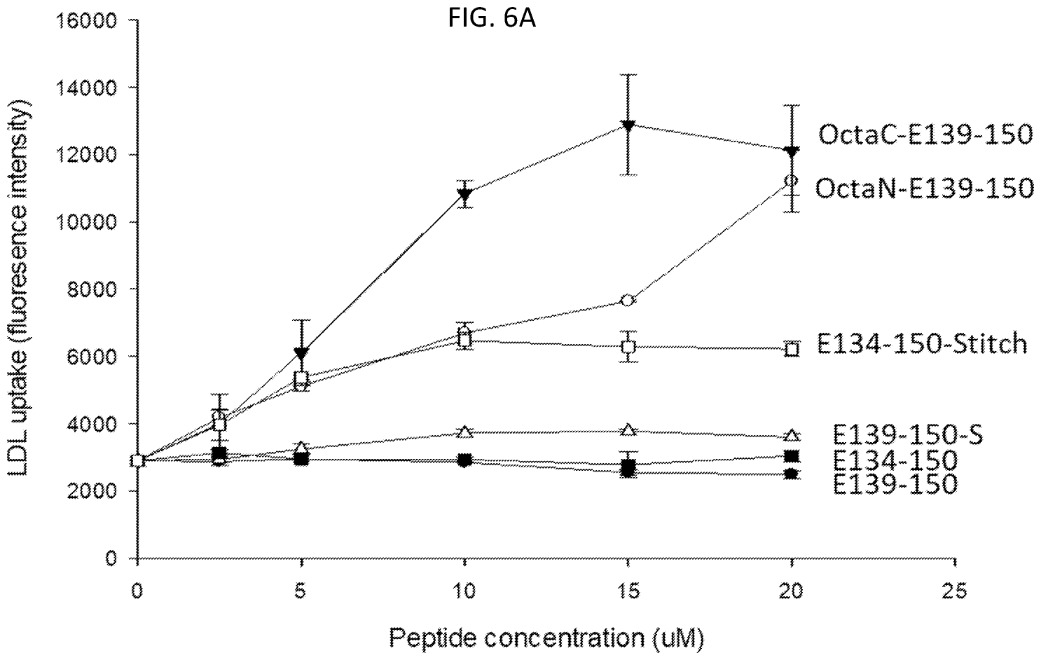

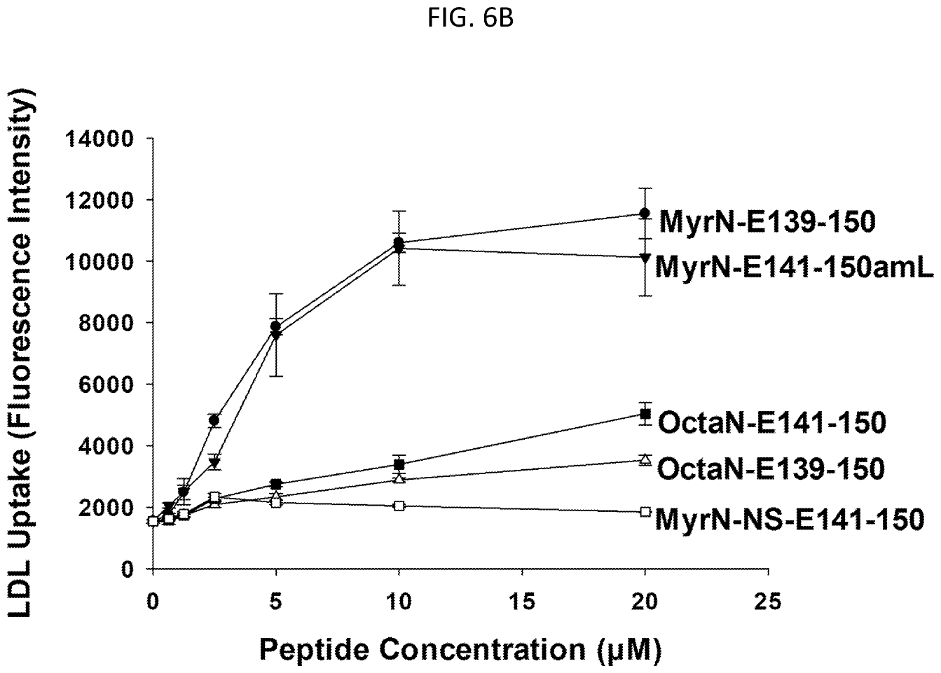

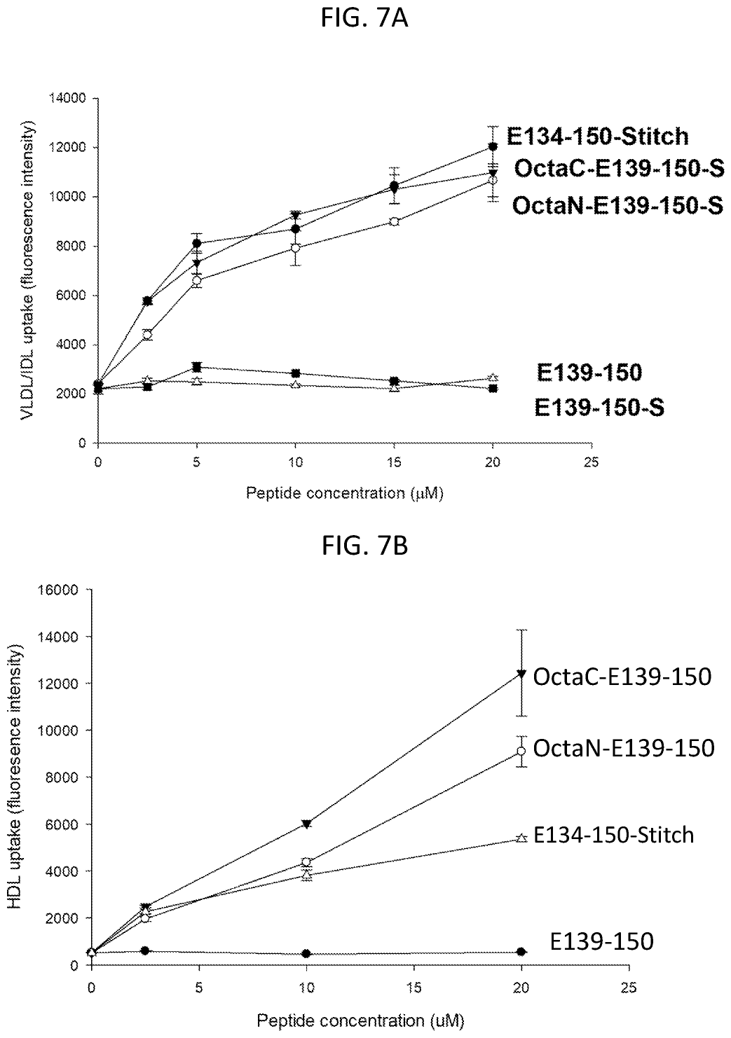

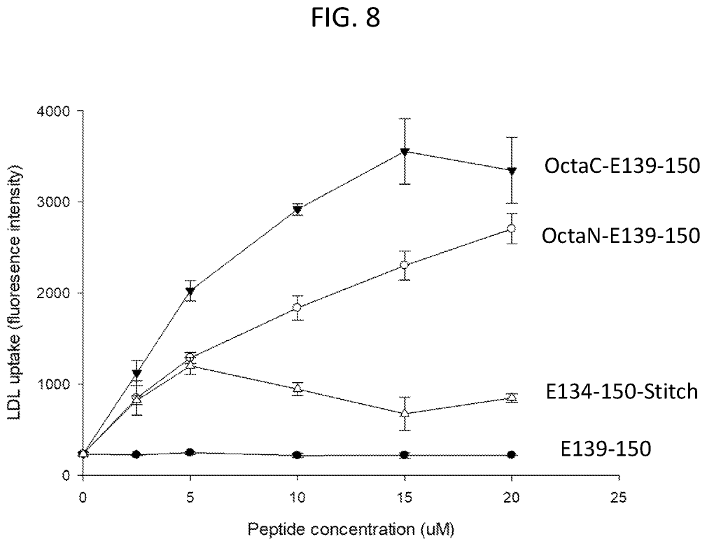

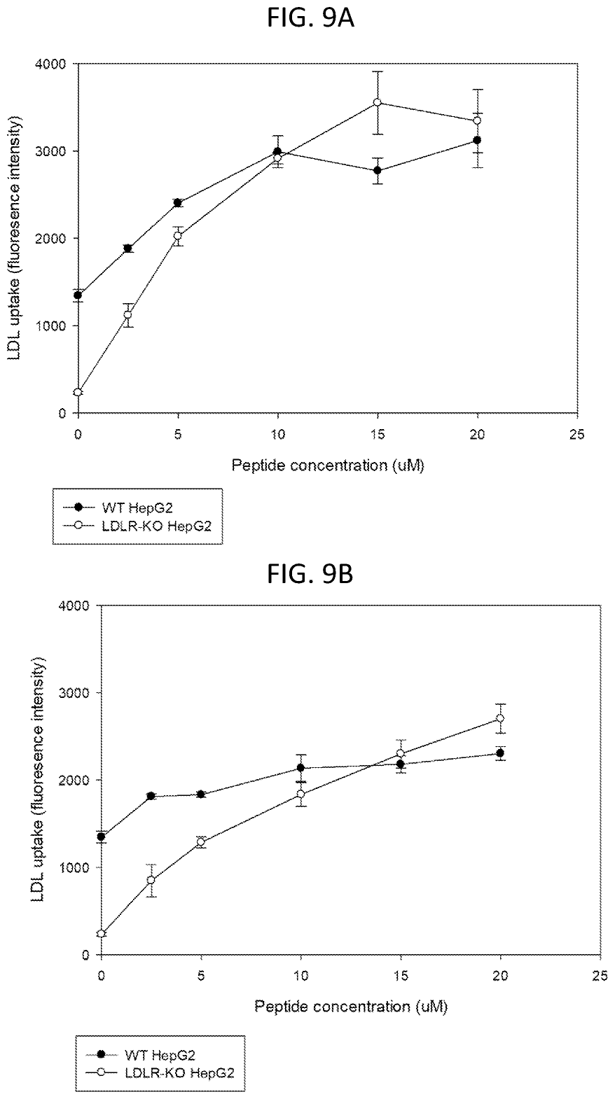

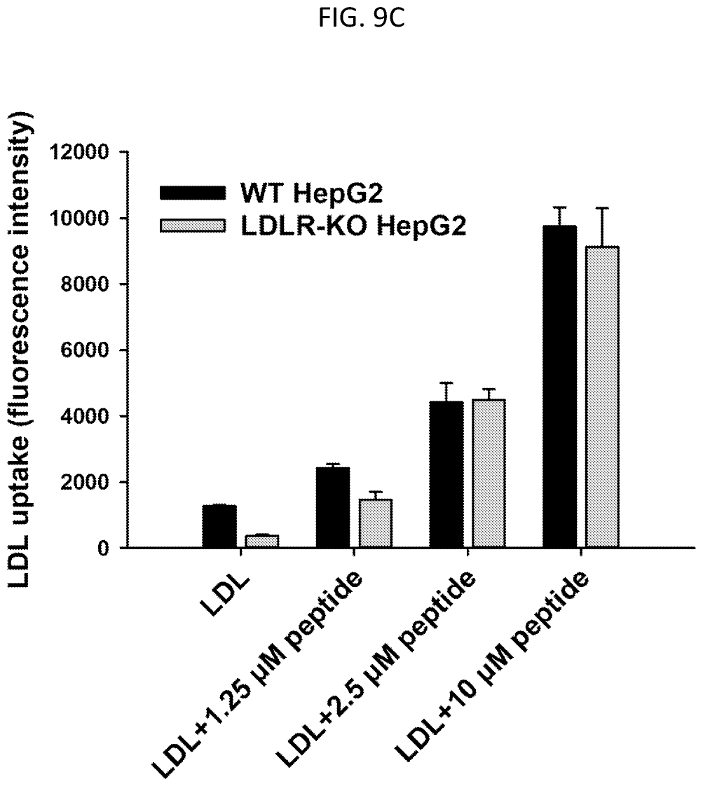

The ApoE mimetic peptides enhanced uptake of LDL in wild type HepG2 cells compared to control peptides (FIGS. 6A and 6B). The peptides also enhanced uptake of VLDL and HDL in wild type HepG2 cells (FIGS. 7A and 7B). At higher concentrations of the peptides, there was an increase in LDL uptake regardless of the presence of LDLR. However, the difference in the uptake at lower peptide concentrations could indicate that apoE peptides also mediate LDL uptake via LDLR pathway (FIG. 8 and FIGS. 9A-9C).

### Example 2

### In Vivo Testing of ApoE Mimetic Peptides

The effect of OctaC-E139-150 and OctaN-E139-150 peptides were tested in ApoE-ko mice. The mice were kept on a normal chow diet. At 3.5 months of age, 15 mice were divided into three groups of five mice each, as follows: 1) control group; 2) OctaC-E139-150 peptide group; and 3) OctaN-E139-150 peptide group. Peptides were dissolved in PBS at 1.5 mg/ml (810 μM OctaC-E139-150; 849 μM OctaN-E139-150). Mice were weighed one day before peptide administration and peptide amount was calculated for each mouse to receive 4.6 mg/kg peptide (2.5 μmol/kg). The calculated amount of 1.5 mg/ml peptide was placed in a syringe and normalized with PBS to 80 μl. The mice received intravenous injection of PBS (control) or the peptide. Blood samples were collected immediately prior to injection, and 1 hour, 3 hours, and 6 hours after injection. Blood was centrifuged at 3000 rpm at 4° C. for 20 minutes and plasma was separated (20-30 μl) and stored at 4° C. Plasma triglycerides and total cholesterol were measured the next day using L-type triglyceride M assay and The cholesterol E kit (Wako, FUJIFILM Medical Systems USA).

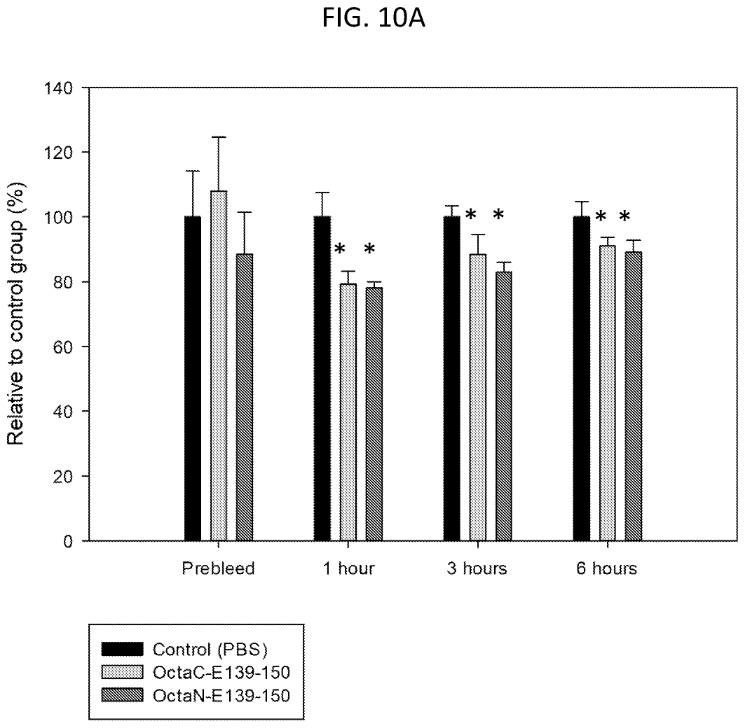

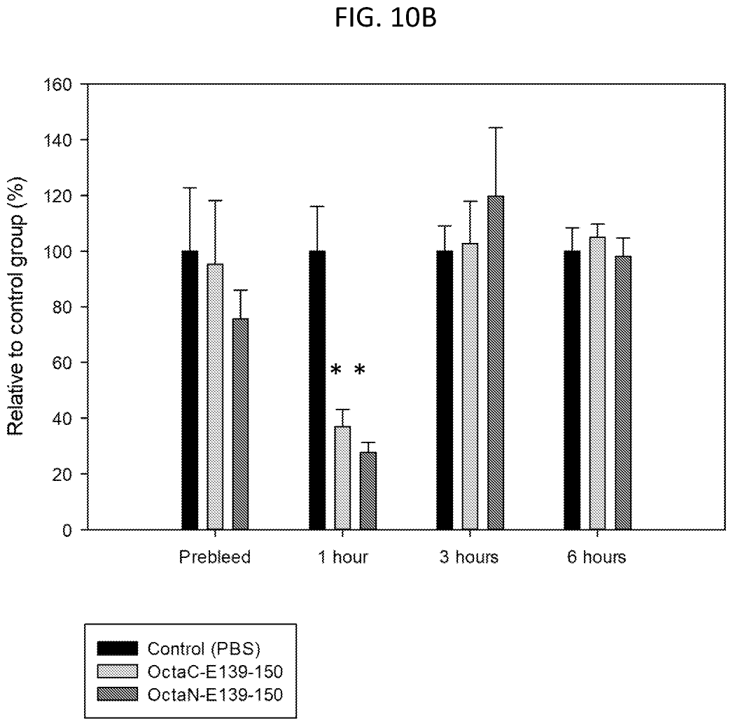

OctaC-E139-150 and OctaN-E139-150 peptides significantly decreased plasma total cholesterol in mice for at least 6 hours after administration (FIG. 10A). In addition, the peptides significantly decreased plasma triglyceride levels, though this effect was transient (FIG. 10B).

### Example 3

### Effect of ApoE Mimetic Peptides on Viral Replication

Mouse Hepatitis Virus (MHV) is a beta coronavirus that infects mice cells via the murine CEACAM1 receptor (mCC1a receptor). HeLa (human epithelium) cells that have been modified to express the mCC1a receptor can be infected with MHV. The recombinant MHV used in this experiment has been modified such that it contains additional nucleocapsid (N) protein segment which is N-terminally fused with a green fluorescence protein tag (GFP) (see Verheije et al., J. Virol. 84:11575-11579, 2010). When cells are infected with MHV, the MHV replicates generating more GFP and increasing the fluorescent signal.

HeLa mCC1a cells were cultured in a 96 well plate. Cells were infected for 2 hours with MHV-GFP at 37° C. Viral stock solution was diluted 40-fold. Stock was prepared by infecting HeLa cells with MHV-GFP for 3 days and by collecting cellular media. Cells were washed with PBS and treated with media (DMEM plus 10% FBS, 100 IU/ml penicillin G, 100 μg/nl streptomycin, 0.5 mg/ml G418) containing ApoE peptides (15 μM) or media without peptide and incubated for 16 hours at 37° C. Cells were washed with PBS and total GFP fluorescence (ex/em 488/520) in cells was measured on the plate using a fluorescence plate reader.

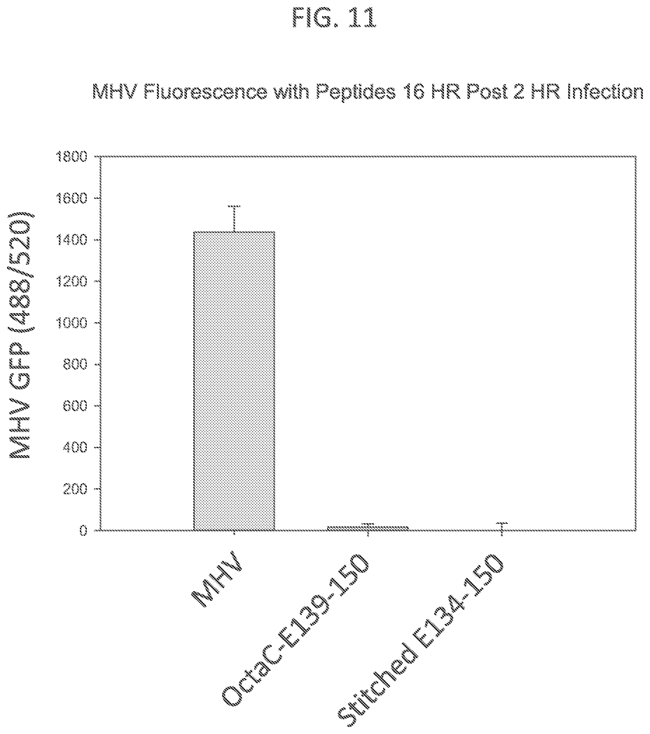

As shown in FIG. 11, treatment with OctaC-E139-150 or stitched E134-150 peptide showed dramatic reductions in MHV-GFP fluorescence, indicating a reduction in viral replication in the treated cells.

### Example 4

### Additional In Vitro Characterization of ApoE Mimetic Peptides

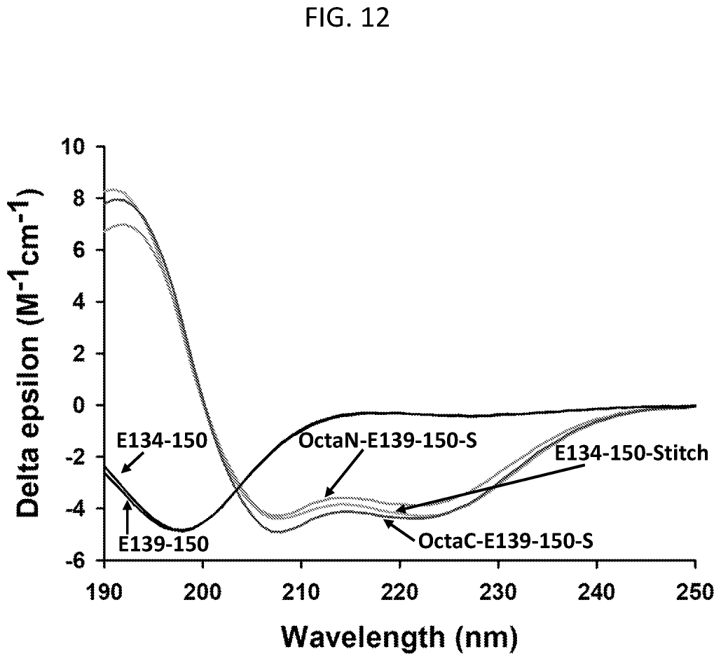

The effect of hydrocarbon stapling on physical properties of ApoE mimetic peptides was studied. Circular dichroism of 100 μM peptides in aqueous solution at 20° C. demonstrated that hydrocarbon-stapled peptides are α-helical, whereas unmodified peptides were disordered (FIG. 12).

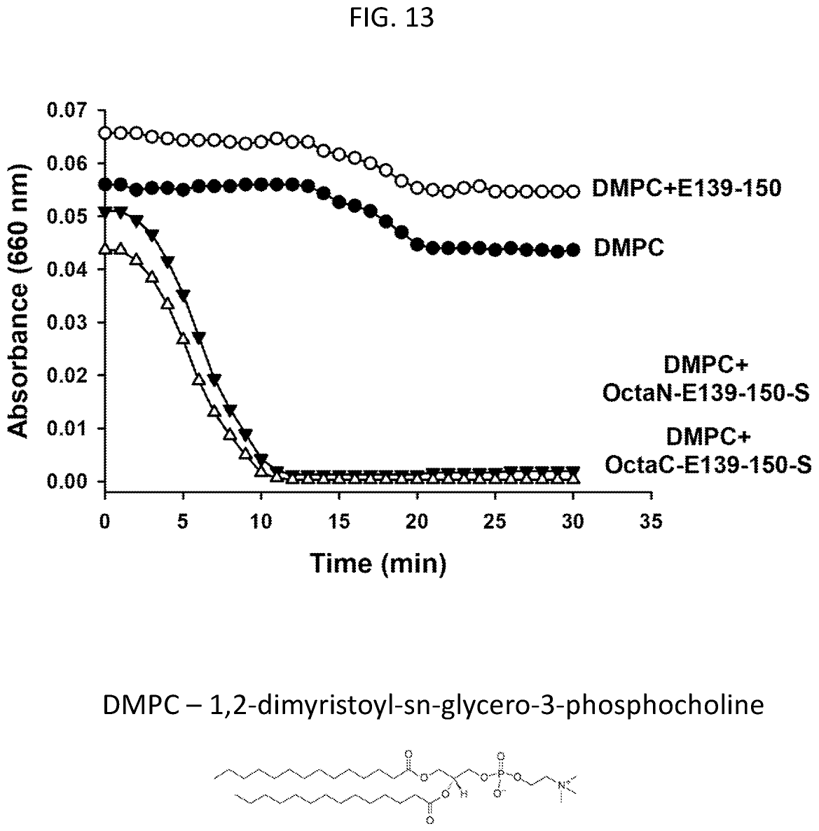

To assess lipid binding properties of unmodified vs acylated stapled peptides, DMPC vesicle solubilization assay was performed. Unmodified linear peptide E139-150 did not solubilize DMPC phospholipid vesicles, whereas acylated hydrocarbon-stapled peptides solubilized DMPC phospholipid vesicles (FIG. 13). This indicates that hydrocarbon stapling and peptide acylation increased lipid binding affinity of the peptides.

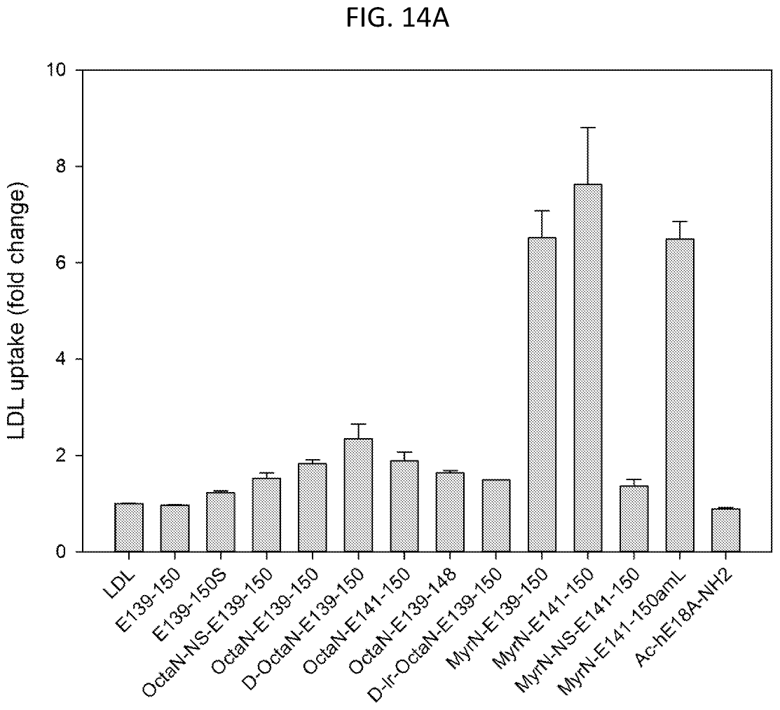

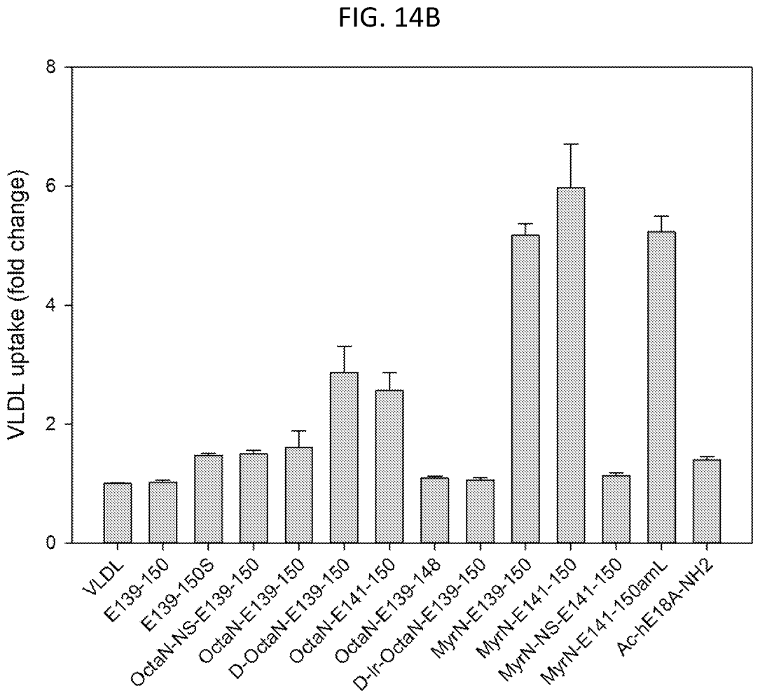

Fluorescently labeled LDL or VLDL (Alexa Fluor 568-NHS ester) at 50 g/ml final protein concentration was preincubated with or without apoE peptides for 30 min at 37° C. Peptide final concentration was 10 μM for LDL uptake and 5 μM for VLDL uptake studies. WT HepG2 cells were then incubated with the lipoprotein/peptide mixtures for 1 h at 37° C. Lipoprotein uptake was assessed by flow cytometry. As shown in FIGS. 14A, 14B, and 6B, hydrocarbon-stapled peptides, and particularly acylated stapled peptides, increased LDL and VLDL uptake by HepG2 cells.

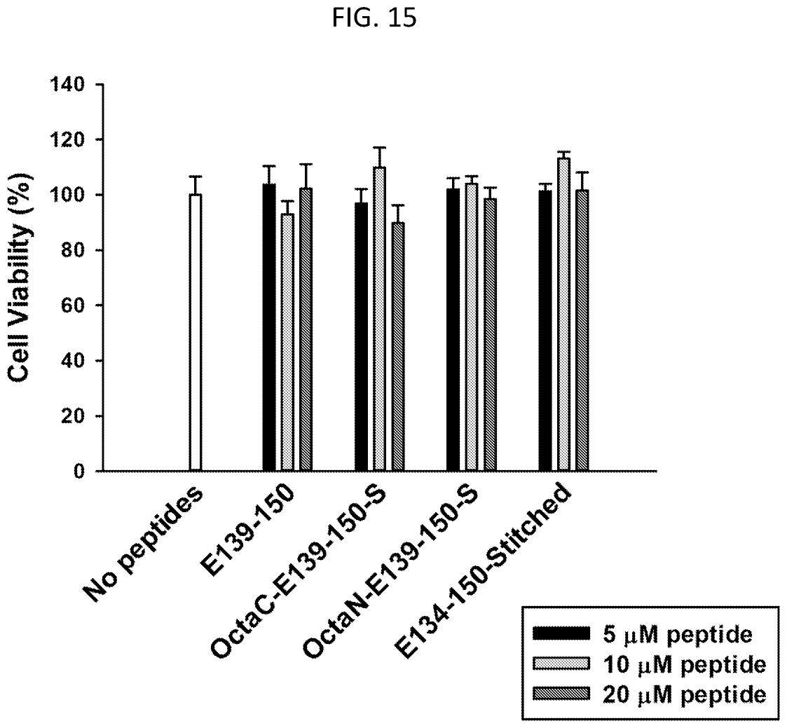

Because cationic amphipathic peptides can be cytotoxic, potential cytotoxic effects of the peptides on the cells were also tested. Cell Counting Kit-8 (Dojindo) was used to assess the effect of increasing concentrations of peptide on wild type HepG2 cell viability. At the conditions used in lipid uptake studies, none of the peptides were cytotoxic to WT HepG2 cells (FIG. 15).

### Example 5

### ApoE Mimetic Peptides Facilitate LDL Binding to Heparin

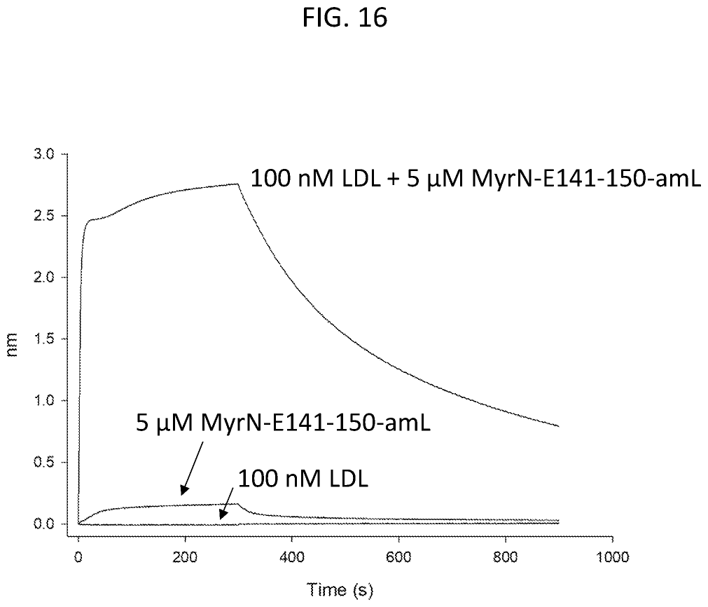

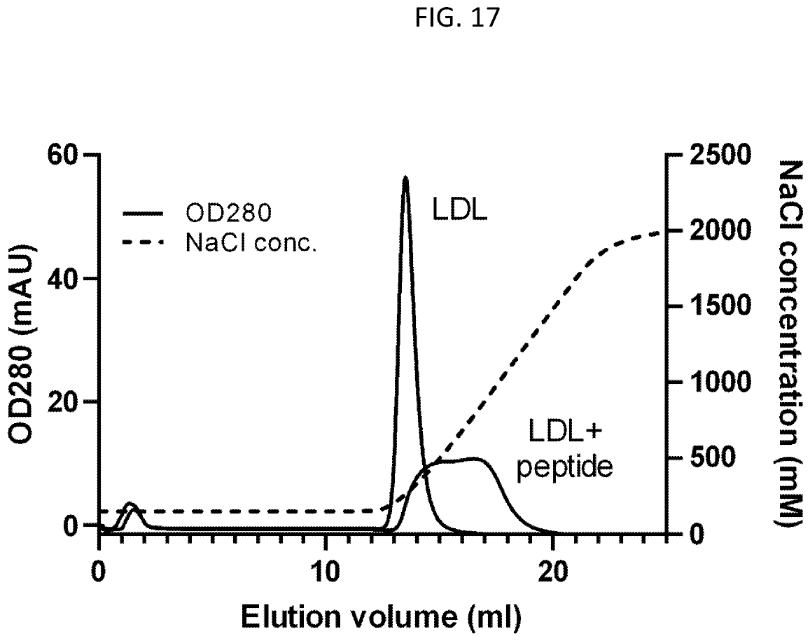

It was observed that the ApoE mimetic peptides facilitate binding of LDL to heparin. This was demonstrated using bio-layer interferometry (BLI) and heparin chromatography. Biotinylated-heparin was immobilized on the BLI Streptavidin (SA) Biosensors (Sartorius). Binding of 100 nM LDL, 5 μM MyrN-E141-150-amL, or precomplexed 100 nM LDL and 5 μM MyrN-E141-150-amL to heparin was measured in PBS, 0.1% BSA (FIG. 16). 400 μl of 1 μM LDL or 1 μM LDL precomplexed with 20 μM MyrN-E141-150-amL was injected to 1 ml HiTrap Heparin HP column. The column was washed with 10 ml of buffer (10 mM sodium phosphate, 0.15 M NaCl, pH 7.4) and LDL or LDL/peptide complex was eluted with linear gradient of NaCl (10 mM sodium phosphate, pH 7.4, 0.15 M-2M NaCl gradient) (FIG. 17).

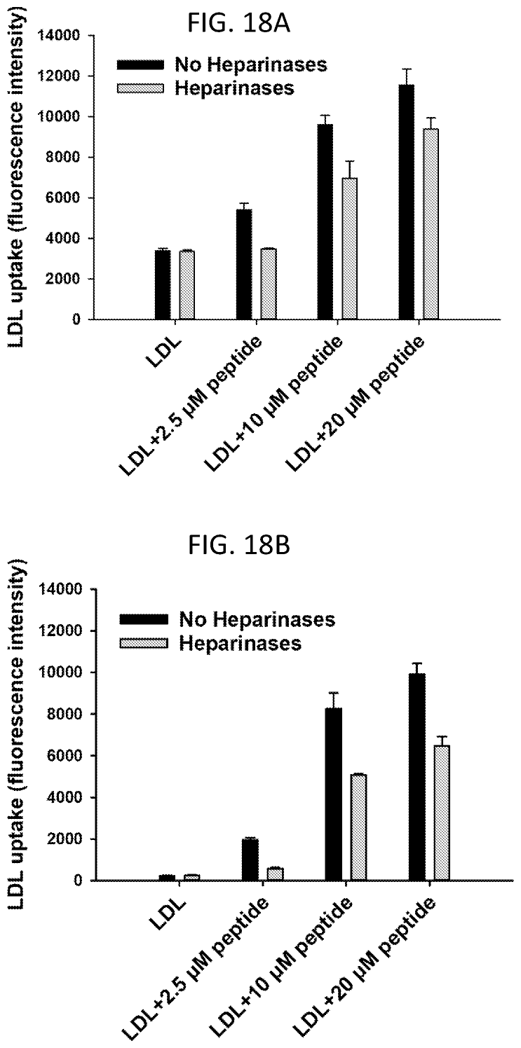

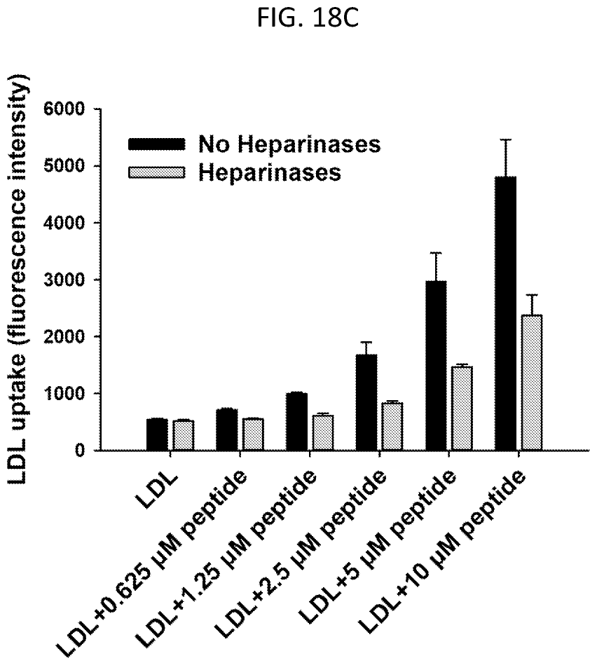

Involvement of heparan sulfate proteoglycans (HSPGs) in peptide-mediated LDL uptake was tested. WT HepG2 cell were pretreated with a mixture of Heparinase I, II, and III (IBEX Pharmaceuticals) to degrade heparan sulfate chains on the cell surface. Next, LDL uptake studies were performed with OctaC-E139-150-S and MyrN-E141-150-amL peptides (FIGS. 18A-18C). LDL uptake without addition of the peptide was not affected by heparinase treatment but heparinase treatment diminished low dose peptide effect on LDL uptake (FIG. 18A). At higher peptide concentrations, heparinase treatment had a lower effect, and some of the uptake was recovered. Similarly, heparinase treatment attenuated MyrN-E141-150-amL mediated LDL uptake in WT HepG2 cells (FIG. 18C). This indicates that HSPGs play a role in peptide mediated uptake of LDL at low peptide concentration, but their effect is lower at high peptide concentration. The same experiments with LDLR-KO HepG2 cells showed similar results (FIG. 18B), indicating that at higher peptide doses, some other receptors, such as LRP1, may be involved in peptide mediated uptake or there is some nonspecific peptide mediated uptake.

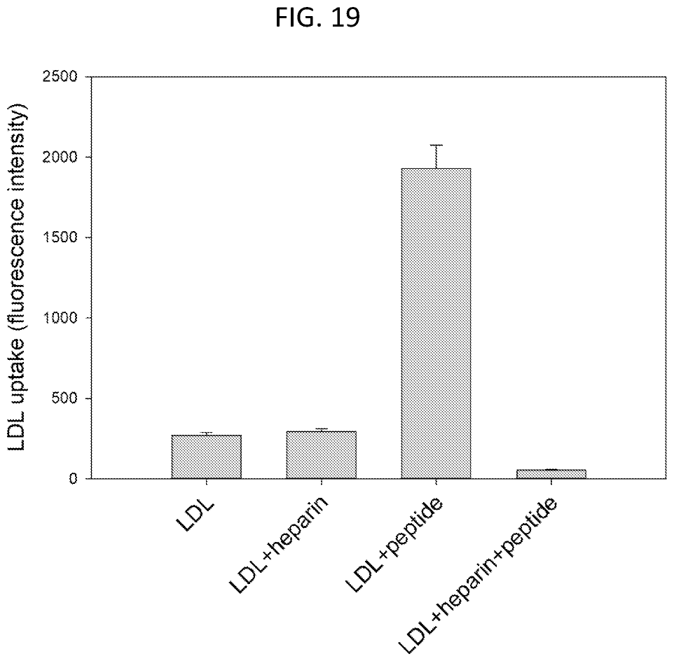

Fluorescently labeled LDL (Alexa Fluor 568-NHS ester) at 50 μg/ml final protein concentration was preincubated with or without 10 μM MyrN-E141-150-amL peptide for 30 min at 37° C. (solutions made in DMEM (no phenol red, no serum) with 0.1% BSA). To some LDL and some LDL/peptide samples, heparin was added (10 USP/ml final conc.; heparin, sodium injection from porcine intestinal mucosa (Fresenius Kabi USA, Lake Zurich, IL)). Mixtures were preincubated for additional 5 min at 37° C. Next, WT HepG2 cells were incubated with the LDL/peptide/heparin mixtures for 1 h at 37° C. Lipoprotein uptake was assessed by flow cytometry. Heparin diminished the effect of MyrN-E141-150-amL peptide on LDL uptake in the wild type HepG2 cells (FIG. 19).

### Example 6

### Additional In Vivo Characterization of ApoE Mimetic Peptides

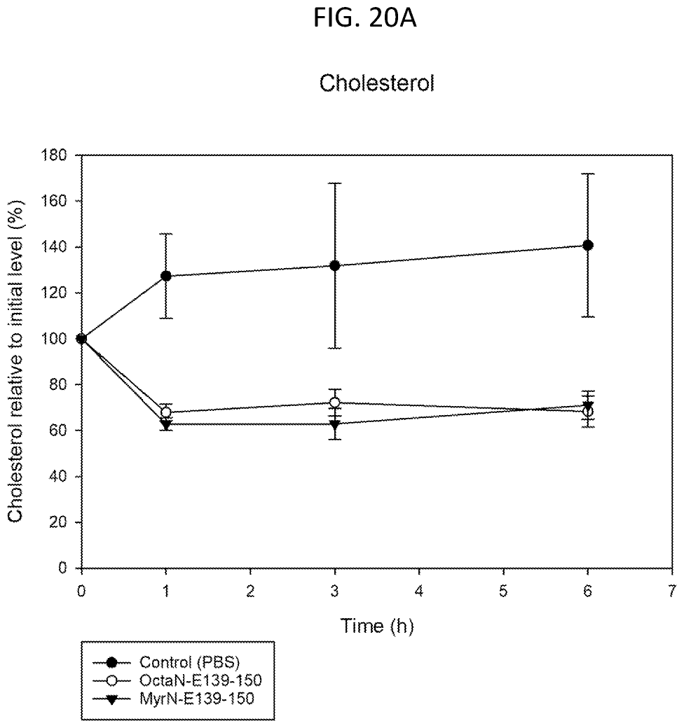

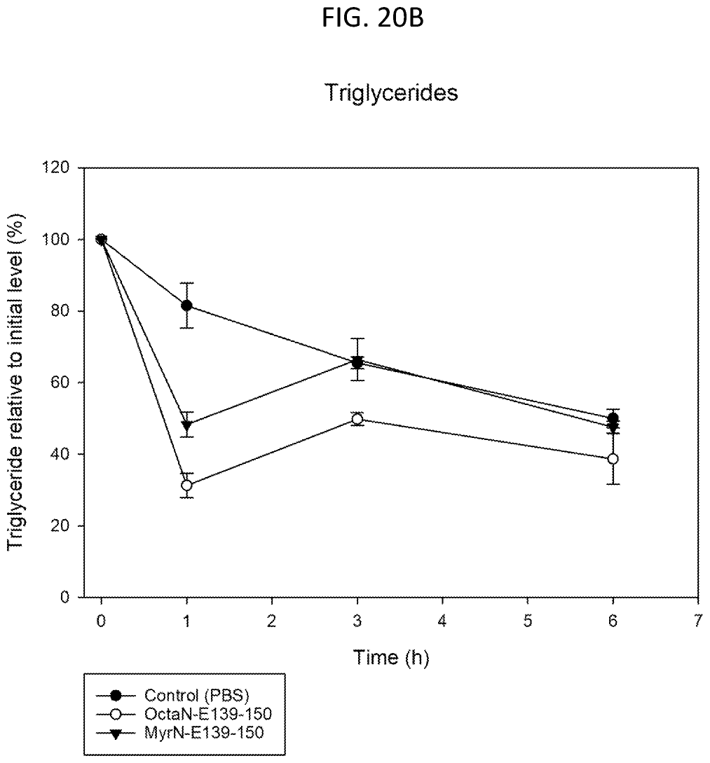

MyrN-E139-150 and OctaN-E139-150 peptides were tested in a single dose ApoE-KO mouse study. ApoE-KO mice (n=9) were divided into 3 groups: control group (PBS), n=3; MyrN-E139-150 (2.5 μmol/kg; 4.6 mg/kg), n=3; and OctaN-E139-150 (2.5 μmol/kg; 4.4 mg/kg), n=3. Mice were fed normal chow diet for the whole study. Peptides were injected i.v. and blood samples were collected at pre-injection and 1 h, 3 h, and 6 h after the injection of the peptides. Measurement of plasma total cholesterol and triglycerides was as described in Example 2. The peptides lowered plasma total cholesterol (FIG. 20A; 37% at 3 h; 29% at 6 h) and triglycerides (FIG. 20B; 30-50% at 1 h).

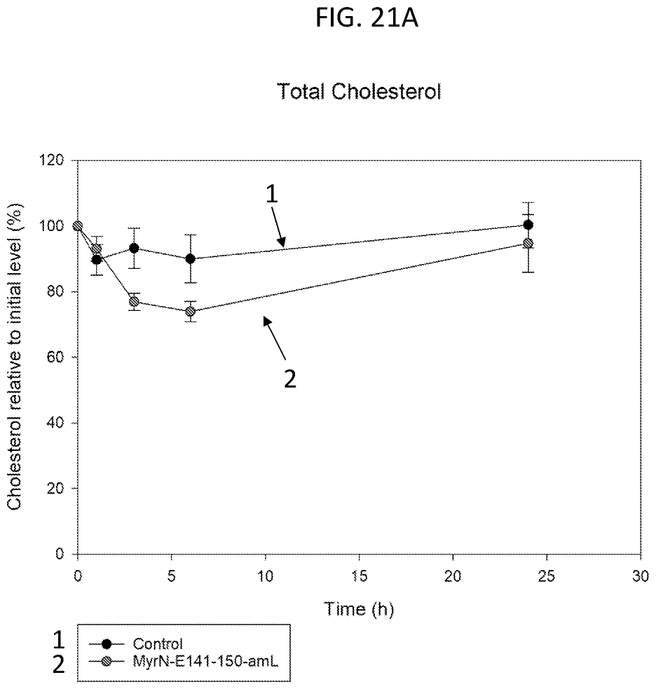

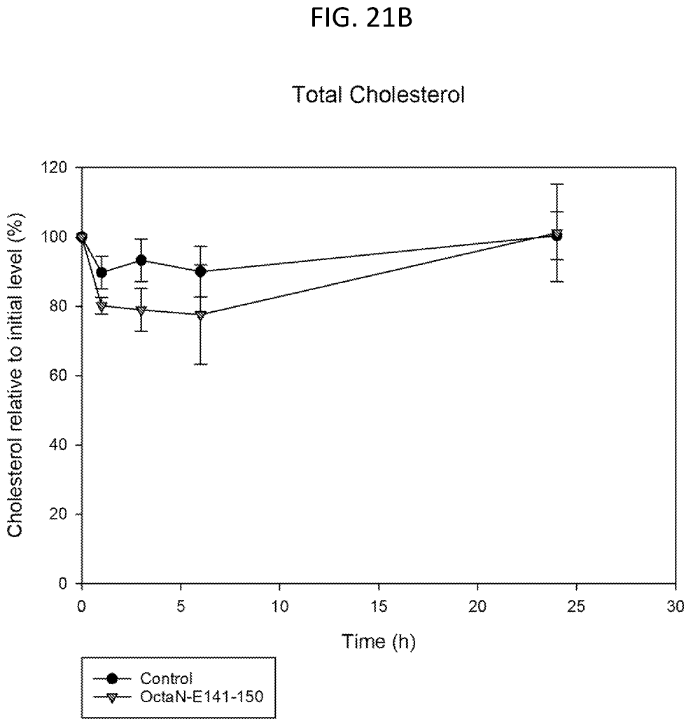

MyrN-E141-150-amL and OctaN-E141-150 were tested in a single dose LDLR-KO mouse study. LDLR-KO mice (n=9) were divided into 3 groups: control group (PBS), n=3; MyrN-E141-150-amL (2.5 μmol/kg; 4.6 mg/kg), n=3; and OctaN-E141-150 (2.5 μmol/kg; 4.4 mg/kg), n=3. Mice were fed normal chow diet for the whole study. Peptides were injected i.v. and blood samples were collected at pre-injection and 1 h, 3 h, 6 h and 24 h after the injection of the peptides. Measurement of plasma total cholesterol was as described in Example 2. MyrN-E141-150-amL lowered plasma total cholesterol around 15-30% after 3 h and 6 h of injection (FIG. 21A).

It will be apparent that the precise details of the methods or compositions described may be varied or modified without departing from the spirit of the described aspects of the disclosure. We claim all such modifications and variations that fall within the scope and spirit of the claims below.

1. An apolipoprotein E (ApoE) mimetic peptide of 8-17 amino acids in length comprising the receptor binding region of ApoE or a portion thereof, comprising one or more covalent linkages joining at least two non-contiguous amino acids of the peptide.

2. The ApoE mimetic peptide of claim 1, wherein the peptide comprises an amphipathic helical domain and the covalent linkage joining at least two non-contiguous amino acids is between two amino acids on the hydrophobic side of the amphipathic helical domain.

3. The ApoE mimetic peptide of claim 1, wherein the one or more covalent linkages is a hydrocarbon staple, a hydrocarbon stitch, a lactam bridge, or a disulfide bond.

4. The ApoE mimetic peptide of claim 3, wherein the hydrocarbon staple or hydrocarbon stitch comprises a linkage comprising one or more of (S)-α-methyl,α-pentenylglycine (S5), (S)-α-methyl,α-octenylglycine (S8), bis-pentenylglycine (B5), (R)-α-methyl,α-pentenylglycine (R5), and (R)-α-methyl,α-octenylglycine (R8).

5. The ApoE mimetic peptide of claim 1, wherein the peptide comprises one or more additional modifications.

6. The ApoE mimetic peptide of claim 5, wherein the modification comprises one or more amino acid substitutions, additions, or deletions; C-terminal amidation; N-terminal acylation; one or more D-isomer amino acids; a modified amino acid; an N-terminal fatty acid; a C-terminal fatty acid; or a combination of two or more thereof, optionally wherein the fatty acid is octanoic acid or myristic acid.

7. (canceled)

8. The ApoE mimetic peptide of claim 1, wherein:
the peptide comprises an amino acid sequence with at least 90% identity to the amino acid sequence of any one of SEQ ID NOs: 31, 2-4, 7-12, 14-21, 30, 32, and 33;
the peptide comprises the amino acid sequence of any one of SEQ ID NOs: 31, 2-4, 7-12, 14-21, 30, 32, and 33; or
the peptide consists of the amino acid sequence of any one of SEQ ID NOs: 31, 2-4, 7-12, 14-21, 30, 32, and 33.

9-10. (canceled)

11. The ApoE mimetic peptide claim 1, wherein the peptide is 8 amino acids long and/or wherein the peptide binds to a lipoprotein.

12. (canceled)

13. The ApoE mimetic peptide of claim 11, wherein the peptide binds to a lipoprotein and the lipoprotein comprises one or more of low density lipoprotein (LDL), high density lipoprotein (HDL), intermediate density lipoprotein (IDL), very low density lipoprotein (VLDL), proteoglycans, LDL receptor (LDLR), and LDLR related protein 1 (LRP1).

14. A fusion protein comprising the ApoE mimetic peptide of claim 1 linked to a human neonatal Fc receptor (FcRn) IgG binding site or FcRn albumin binding site.

15. The fusion protein of claim 14, wherein the fusion protein:
comprises an amino acid sequence with at least 90% identity to the amino acid sequence of any one of SEQ ID NOs: 22-25;
comprises the amino acid sequence of any one of SEQ ID NOs: 22-25; or
consists of the amino acid sequence of any one of SEQ ID NOs: 22-25.

16-17. (canceled)

18. A pharmaceutical composition comprising:
the ApoE mimetic peptide of claim 1 or a fusion protein comprising the peptide and a human neonatal Fc receptor (FcRn) IgG binding site or FcRn albumin binding site; and
a pharmaceutically acceptable carrier.

19. The pharmaceutical composition of claim 18, further comprising a phospholipid.

20. The pharmaceutical composition of claim 19, wherein the phospholipid comprises 1, 2-dimyristoyl-sn-glycero-3-phosphocholine.

21. The pharmaceutical composition of claim 18, wherein the composition is formulated for intravenous administration, subcutaneous administration, or oral administration.

22. A method of treating a dyslipidemic disorder in a subject, comprising administering to the subject an effective amount of the composition of claim 18.

23. The method of claim 22, wherein the method reduces triglyceride levels in the subject.

24. The method of claim 23, wherein the subject has hypertriglyceridemia and/or a pre-treatment serum triglyceride level of 150 mg/dL or more.

25. (canceled)

26. The method of claim 22, wherein the method reduces total cholesterol levels in the subject.

27. The method of claim 26, wherein the subject has hypercholesterolemia and/or a pre-treatment total cholesterol level of 200 mg/dL or more.

28. (canceled)

29. A method of treating a viral infection in a subject, comprising administering to the subject an effective amount of the composition of claim 18, thereby treating the viral infection.

30. The method of claim 29, wherein the viral infection is infection with an enveloped virus, a betacoronavirus, or a severe acute respiratory syndrome coronavirus 2 (SARS-CoV-2).

31-32. (canceled)

33. The method of claim 29, wherein treating the viral infection comprises inhibiting replication of the virus in the subject.

34. The method of claim 22, wherein the administering comprises intravenous administration, subcutaneous injection, oral administration, nasal administration, or aerosol administration.

35. (canceled)

36. A method of making the ApoE mimetic peptide of claim 1 or a fusion protein comprising the peptide and a human neonatal Fc receptor (FcRn) IgG binding site or FcRn albumin binding site, comprising producing the peptide or fusion protein recombinantly and/or by chemical synthesis.

37. (canceled)

38. A method of reducing lipoproteins in a sample, comprising:
contacting a sample containing lipoproteins with one or more of the ApoE mimetic peptides of claim 1 under conditions sufficient for binding of lipoproteins to the one or more peptides, thereby forming peptide/lipoprotein complexes;
contacting the peptide/lipoprotein complexes with glycosaminoglycans under conditions sufficient for binding of the peptide/lipoprotein complexes to the glycosaminoglycans, thereby forming peptide/lipoprotein/glycosaminoglycan complexes; and
removing the peptide/lipoprotein/glycosaminoglycan complexes from the sample.

39. The method of claim 38, wherein the sample comprises plasma.

40. The method of claim 38, wherein the sample is from a subject with familial hypercholesteremia.

41. The method of claim 38, wherein the glycosaminoglycan is immobilized on a solid support and/or the method is performed with an apheresis system.

42. (canceled)

43. The method of claim 38, wherein the glycosaminoglycan is dextran sulfate, heparin, or heparan sulfate.


SEQUENCE LISTING (37 sequences)

SEQ ID NO: 1 - protein, 12 aa, synthetic construct
  MOD_RES 1: note = acylation
  MOD_RES 12: note = amidation
SHLRKLRKRLLR

SEQ ID NO: 2 - protein, 12 aa, synthetic construct
  MOD_RES 1: note = acylation
  MOD_RES 3: note = X is (R)-alpha-methyl,alpha-octenylglycine
  MOD_RES 10: note = X is (S)-alpha-methyl,alpha-pentenylglycine
  MOD_RES 12: note = amidation
SHXRKLRKRXLR

SEQ ID NO: 3 - protein, 12 aa, synthetic construct
  MOD_RES 1: note = octanoyl
  MOD_RES 3: note = X is(R)-alpha-methyl,alpha-octenylglycine
  MOD_RES 10: note = X is (S)-alpha-methyl,alpha-pentenylglycine
  MOD_RES 12: note = amidation
SHXRKLRKRXLR

SEQ ID NO: 4 - protein, 12 aa, synthetic construct
  MOD_RES 1: note = acylation
  MOD_RES 3: note = X is (R)-alpha-methyl,alpha-octenylglycine
  MOD_RES 10: note = X is (S)-alpha-methyl,alpha-pentenylglycine
  MOD_RES 12: note = octanoyl
SHXRKLRKRXLR

SEQ ID NO: 5 - protein, 12 aa, synthetic construct
  MOD_RES 1: note = octanoyl
  MOD_RES 12: note = amidation
SHLRKLRKRLLR

SEQ ID NO: 6 - protein, 12 aa, synthetic construct
  MOD_RES 1: note = acylation
  MOD_RES 12: note = octanoyl
SHLRKLRKRLLR

SEQ ID NO: 7 - protein, 12 aa, synthetic construct
  MOD_RES 1: note = acylation
  MOD_RES 3: note = X is (S)-alpha-methyl,alpha-octenylglycine
  MOD_RES 10: note = X is (R)-alpha-methyl,alpha-pentenylglycine
  MOD_RES 12: note = octanoyl
  SITE 1: note = D-serine
  SITE 2: note = D-histidine
  SITE 4: note = D-arginine
  SITE 5: note = D-lysine
  SITE 6: note = D-leucine
  SITE 7: note = D-arginine
  SITE 8: note = D-lysine
  SITE 9: note = D-arginine
  SITE 11: note = D-leucine
  SITE 12: note = D-argninine
SHXRKLRKRXLR

SEQ ID NO: 8 - protein, 12 aa, synthetic construct
  MOD_RES 1: note = octanoyl
  MOD_RES 3: note = X is (S)-alpha-methyl,alpha-octenylglycine
  MOD_RES 10: note = X is (R)-alpha-methyl,alpha-pentenylglycine
  MOD_RES 12: note = amidation
  SITE 1: note = D-serine
  SITE 2: note = D-histidine
  SITE 4: note = D-arginine
  SITE 5: note = D-lysine
  SITE 6: note = D-leucine
  SITE 7: note = D-arginine
  SITE 8: note = D-lysine
  SITE 9: note = D-arginine
  SITE 11: note = D-leucine
  SITE 12: note = D-arginine
SHXRKLRKRXLR

SEQ ID NO: 9 - protein, 12 aa, synthetic construct
  MOD_RES 1: note = acylation
  MOD_RES 3: note = X is (R)-alpha-methyl,alpha-octenylglycine
  MOD_RES 10: note = X is (S)-alpha-methyl,alpha-pentenylglycine
  MOD_RES 12: note = octanoyl
  SITE 11: note = D-leucine
  SITE 12: note = D-arginine
SHXRKLRKRXLR

SEQ ID NO: 10 - protein, 12 aa, synthetic construct
  MOD_RES 1: note = octanoyl
  MOD_RES 3: note = X is (R)-alpha-methyl,alpha-octenylglycine
  MOD_RES 10: note = X is (S)-alpha-methyl,alpha-pentenylglycine
  MOD_RES 12: note = amidation
  SITE 11: note = D-leucine
  SITE 12: note = D-arginine
SHXRKLRKRXLR

SEQ ID NO: 11 - protein, 12 aa, synthetic construct
  MOD_RES 1: note = acylation
  MOD_RES 3: note = X is (R)-alpha-methyl,alpha-octenylglycine
  MOD_RES 10: note = X is (S)-alpha-methyl,alpha-pentenylglycine
  MOD_RES 12: note = myristyl
SHXRKLRKRXLR

SEQ ID NO: 12 - protein, 12 aa, synthetic construct
  MOD_RES 1: note = myristyl
  MOD_RES 12: note = amidation
  MOD_RES 3: note = X is (R)-alpha-methyl,alpha-octenylglycine
  MOD_RES 10: note = X is (S)-alpha-methyl,alpha-pentenylglycine
SHXRKLRKRXLR

SEQ ID NO: 13 - protein, 17 aa, synthetic construct
  MOD_RES 1: note = acylation
  MOD_RES 17: note = amidation
RVRLASHLRKLRKRLLR

SEQ ID NO: 14 - protein, 17 aa, synthetic construct
  MOD_RES 1: note = acylation
  MOD_RES 2: note = X is (S)-alpha-methyl,alpha-pentenylglycine
  MOD_RES 6: note = X is (S)-alpha-methyl,alpha-pentenylglycine
  MOD_RES 8: note = X is R8: (R)-alpha-methyl,alpha-octenylglycine
  MOD_RES 15: note = X is (S)-alpha-methyl,alpha-pentenylglycine
  MOD_RES 17: note = amidation
RXRLAXHXRKLRKRXLR

SEQ ID NO: 15 - protein, 17 aa, synthetic construct
  MOD_RES 1: note = acylation
  MOD_RES 4: note = X is (S)-alpha-methyl,alpha-pentenylglycine
  MOD_RES 8: note = X is bis-pentenylglycine
  MOD_RES 15: note = X is (S)-alpha-methyl,alpha-octenylglycine
  MOD_RES 17: note = amidation
RVRXASHXRKLRKRXLR

SEQ ID NO: 16 - protein, 10 aa, synthetic construct
  MOD_RES 1: note = octanoyl
  MOD_RES 1: note = X is (R)-alpha-methyl,alpha-octenylglycine
  MOD_RES 8: note = X is (S)-alpha-methyl,alpha-pentenylglycine
  MOD_RES 10: note = amidation
XRKLRKRXLR

SEQ ID NO: 17 - protein, 10 aa, synthetic construct
  MOD_RES 1: note = acylation
  MOD_RES 1: note = X is (R)-alpha-methyl,alpha-octenylglycine
  MOD_RES 8: note = X is (S)-alpha-methyl,alpha-pentenylglycine
  MOD_RES 10: note = octanoyl
XRKLRKRXLR

SEQ ID NO: 18 - protein, 10 aa, synthetic construct
  MOD_RES 1: note = octanoyl
  MOD_RES 3: note = X is (R)-alpha-methyl,alpha-octenylglycine
  MOD_RES 10: note = X is (S)-alpha-methyl,alpha-pentenylglycine
  MOD_RES 10: note = amidation
SHXRKLRKRX

SEQ ID NO: 19 - protein, 10 aa, synthetic construct
  MOD_RES 1: note = acylation
  MOD_RES 3: note = X is (R)-alpha-methyl,alpha-octenylglycine
  MOD_RES 10: note = X is (S)-alpha-methyl,alpha-pentenylglycine
  MOD_RES 10: note = octanoyl
SHXRKLRKRX

SEQ ID NO: 20 - protein, 8 aa, synthetic construct
  MOD_RES 1: note = octanoyl
  MOD_RES 1: note = X is (R)-alpha-methyl,alpha-octenylglycine
  MOD_RES 8: note = X is (S)-alpha-methyl,alpha-pentenylglycine
  MOD_RES 8: note = amidation
XRKLRKRX

SEQ ID NO: 21 - protein, 8 aa, synthetic construct
  MOD_RES 1: note = acylation
  MOD_RES 1: note = X is R8: (R)-alpha-methyl,alpha-octenylglycine
  MOD_RES 8: note = X is (S)-alpha-methyl,alpha-pentenylglycine
  MOD_RES 8: note = octanoyl
XRKLRKRX

SEQ ID NO: 22 - protein, 26 aa, synthetic construct
  MOD_RES 1: note = acylation
  MOD_RES 3: note = X is penicillamine
  MOD_RES 9: note = X is N-methyl-Glycine
  MOD_RES 10: note = X is N-methyl-Leucine
  MOD_RES 14: note = X is 6-aminohexanoic acid
  MOD_RES 17: note = X is (R)-alpha-methyl,alpha-octenylglycine
  MOD_RES 24: note = X is (S)-alpha-methyl,alpha-pentenylglycine
  MOD_RES 26: note = octanoyl
RFXTGHFGXXYPCXSHXRKLRKRXLR

SEQ ID NO: 23 - protein, 26 aa, synthetic construct
  MOD_RES 1: note = octanoyl
  MOD_RES 3: note = X is (R)-alpha-methyl,alpha-octenylglycine
  MOD_RES 10: note = X is (S)-alpha-methyl,alpha-pentenylglycine
  MOD_RES 13: note = X is 6-aminohexanoic acid
  MOD_RES 16: note = X is penicillamine
  MOD_RES 22: note = X is N-methyl-Glycine
  MOD_RES 23: note = X is N-methyl-Leucine
SHXRKLRKRXLRXRFXTGHFGXXYPC

SEQ ID NO: 24 - protein, 28 aa, synthetic construct
  MOD_RES 1: note = acylation
  MOD_RES 16: note = X is 6-aminohexanoic acid
  MOD_RES 19: note = X is(R)-alpha-methyl,alpha-octenylglycine
  MOD_RES 26: note = X is (S)-alpha-methyl,alpha-pentenylglycine
  MOD_RES 28: note = octanoyl
VMHCFWDEEFKCDYGXSHXRKLRKRXLR

SEQ ID NO: 25 - protein, 28 aa, synthetic construct
  MOD_RES 1: note = octanoyl
  MOD_RES 3: note = X is (R)-alpha-methyl,alpha-octenylglycine
  MOD_RES 10: note = X is (S)-alpha-methyl,alpha-pentenylglycine
  MOD_RES 13: note = X is 6-aminohexanoic acid
  MOD_RES 28: note = amidation
SHXRKLRKRXLRXVMHCFWDEEFKCDYG

SEQ ID NO: 26 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 27 - protein, 14 aa, synthetic construct
  MOD_RES 3: note = X is penicillamine
  MOD_RES 9: note = X is N-methyl-Glycine
  MOD_RES 10: note = X is N-methyl-Leucine
  MOD_RES 14: note = Xis 6-aminohexanoic acid
RFXTGHFGXXYPCX

SEQ ID NO: 28 - protein, 16 aa, synthetic construct
  MOD_RES 16: note = X is 6-aminohexanoic acid
VMHCFWDEEFKCDYGX

SEQ ID NO: 29 - protein, 10 aa, synthetic construct
  MOD_RES 1: note = myristyl
  MOD_RES 10: note = amidation
LRKLRKRLLR

SEQ ID NO: 30 - protein, 10 aa, synthetic construct
  MOD_RES 1: note = myristyl
  MOD_RES 1: note = X is (R)-alpha-methyl,alpha-octenylglycine
  MOD_RES 8: note = X is (S)-alpha-methyl,alpha-pentenylglycine
  MOD_RES 10: note = amidation
XRKLRKRXLR

SEQ ID NO: 31 - protein, 10 aa, synthetic construct
  MOD_RES 1: note = myristyl
  MOD_RES 1: note = X is (R)-alpha-methyl,alpha-octenylglycine
  MOD_RES 8: note = X is (S)-alpha-methyl,alpha-pentenylglycine
  MOD_RES 9: note = X is alpha-methyl-Leucine
  MOD_RES 10: note = amidation
XRKLRKRXXR

SEQ ID NO: 32 - protein, 10 aa, synthetic construct
  MOD_RES 1: note = octanoyl
  MOD_RES 1: note = X is (S)-alpha-methyl,alpha-octenylglycine
  SITE 2: note = D-arginine
  SITE 3: note = D-lysine
  SITE 4: note = D-leucine
  SITE 5: note = D-arginine
  SITE 6: note = D-lysine
  SITE 7: note = D-arginine
  MOD_RES 8: note = X is (R)-alpha-methyl,alpha-pentenylglycine
  SITE 9: note = D-leucine
  SITE 10: note = D-arginine
  MOD_RES 10: note = amidation
XRKLRKRXLR

SEQ ID NO: 33 - protein, 10 aa, synthetic construct
  MOD_RES 1: note = myristyl
  MOD_RES 1: note = X is (S)-alpha-methyl,alpha-octenylglycine
  SITE 2: note = D-arginine
  SITE 3: note = D-lysine
  SITE 4: note = D-leucine
  SITE 5: note = D-arginine
  SITE 6: note = D-lysine
  SITE 7: note = D-arginine
  MOD_RES 8: note = X is (R)-alpha-methyl,alpha-pentenylglycine
  SITE 9: note = D-leucine
  SITE 10: note = D-arginine
  MOD_RES 10: note = amidation
XRKLRKRXLR

SEQ ID NO: 34 - protein, 17 aa, synthetic construct
RVRLASHLRKLRKRLLR

SEQ ID NO: 35 - protein, 12 aa, synthetic construct
SHLRKLRKRLLR

SEQ ID NO: 36 - protein, 10 aa, synthetic construct
LRKLRKRLLR

SEQ ID NO: 37 - protein, 8 aa, synthetic construct
LRKLRKRL